(19) United States
(12) Patent Application Publication
(10) Pub. No.: US 2026/0000713 A1
(43) Pub. Date: Jan. 1, 2026

(54) MICROBIAL METABOLITES ON INTESTINAL INFLAMMATION

(71) Applicants: CEDARS-SINAI MEDICAL CENTER, Los Angeles, CA (US); THE REGENTS OF THE UNIVERSITY OF CALIFORNIA, Oakland, CA (US); UNITED STATES OF AMERICA AS REPRESENTED BY THE DEPARTMENT OF VETERANS AFFAIRS

(72) Inventors: Noam JACOB, Los Angeles, CA (US); Stephan R. TARGAN, Santa Monica, CA (US); Kathrin S. MICHELSEN, Los Angeles, CA (US); Erica ALEXEEV, Los Angeles, CA (US)

(21) Appl. No.: 18/715,557

(22) PCT Filed: Nov. 30, 2022
(86) PCT No.: PCT/US2022/051440

§ 371 Date: May 31, 2024

Related U.S. Application Data
(60) Provisional application No. 63/285,058, filed on Dec. 1, 2021.

(51) Int. Cl. A61K 35/74; A61K 45/06; C12Q 1/06; C12Q 1/6883; G01N 33/53; G01N 33/68

(52) U.S. Cl. CPC A61K 35/74; A61K 45/06; C12Q 1/06; C12Q 1/6883; G01N 33/5308; G01N 33/6863; C12Q 2600/118; C12Q 2600/156; C12Q 2600/158; G01N 2333/57; G01N 2800/065; G01N 2800/50

(57) ABSTRACT

Provided herein are methods of predicting a likelihood that a subject will develop ileitis, intestinal fibrosis or colitis based on an amount of acetate or acetate-producing bacteria detected in a sample obtained from a subject. Certain genetic risk variants at TNF super-family member 15 (TNFSF15) may also be detected. Methods, systems and kits for treatment of develop ileitis, intestinal fibrosis or colitis are also provided, which include inhibitors of acetate or acetate-producing bacteria, or targeting biologic therapeutics, such as inhibitors of Tumor necrosis factor (TNF)-like cytokine 1A (TL1A).

### CROSS-REFERENCE

This application claims the benefit of U.S. Provisional Application No. 63/285,058, filed Dec. 1, 2021, which application is herein incorporated by reference in its entirety.

### STATEMENT AS TO FEDERALLY SPONSORED RESEARCH

This invention was made with government support under Grant No. DK 123511 awarded by the National Institutes of Health, and Grant No. and 1IK2CX002157 awarded by the United States Department of Veterans Affairs. The government has certain rights in the invention.

### SUMMARY

In certain aspects, disclosed herein is a method of treating inflammatory bowel disease (IBD) in a subject, the method comprising: administering to the subject an active agent effective to reduce an amount of acetate or a metabolite thereof, or acetate producing bacteria in the subject, wherein the subject is predicted to develop ileitis, intestinal fibrosis, or colitis based, at least in part, on an increase in an amount of the acetate or the metabolite thereof, or an amount of the acetate producing bacteria, relative to a reference level in a reference subject that does not have the IBD detected in one or more samples obtained from the subject. In some embodiments, the active agent is effective to reduce the amount of the acetate producing bacteria in the subject, wherein the active agent comprises a small molecule drug, an antibody or antigen-binding fragment thereof, an inhibitor, or an enzyme or catalytically active portion thereof. In some embodiments, the active agent comprises an antibiotic, a probiotic, a prebiotic, or any combination thereof. In some embodiments, the active agent comprises an antibiotic. In some embodiments, the active agent comprises a probiotic. In some embodiments, the active agent comprises a prebiotic. In some embodiments, the probiotic is administered concurrently with the antibiotic. In some embodiments, the probiotic is administered following the antibiotic. In some embodiments, the probiotic is administered concurrently with the prebiotic. In some embodiments, the active agent comprises a fecal microbiota transplant to reduce an amount of the acetate producing bacteria in the subject. In some embodiments, the fecal microbiota transplant is colonically administered. In some embodiments, the fecal microbiota transplant comprises bacteria that do not produce acetate in an amount exceeding 50 mmol/L. In some embodiments, the fecal microbiota transplant comprises a supplement derived from a stool sample from an individual that does not have the IBD. In some embodiments, the active agent is effective to reduce the amount of the acetate or the metabolite thereof, wherein the active agent comprises a small molecule drug, an antibody or antigen-binding fragment thereof, an inhibitor, or an enzyme or catalytically active portion thereof. In some embodiments, the active agent comprises a chelating agent. In some embodiments, the chelating agent is colonically administered. In some embodiments, the chelating agent is configured to bind acetate. In some embodiments, the active agent comprises a chelating agent configured to bind the metabolite of acetate. In some embodiments, active agent comprises a chelating agent configured to bind 3-hydroxy-propionate. In some embodiments, the active agent comprises an inhibitor. In some embodiments, the active agent comprises antagonists of acetate receptor (GPR43). In some embodiments, the small molecule drug is therapeutically effective to disrupt the production of the acetate or the metabolite thereof in the subject by the acetate producing bacteria. In some embodiments, the antibody or antigen binding fragment thereof is therapeutically effective to bind to the acetate or the metabolite thereof. In some embodiments, the enzyme or the catalytically active portion thereof promotes the metabolism of acetate or the metabolite thereof in the subject. In some embodiments, the enzyme comprises coenzyme A, acetyl-coenzyme A, formate acetyl transferase, or any combination thereof. In some embodiments, the method comprises prescribing a change in a diet of the subject. In some embodiments, changing the subject's diet comprises reducing the subject's intake of acid food, vinegar, acetic acid, carbohydrates, sugars, starches, probiotic-containing foods, kim chi, kombucha, or any combination thereof. In some embodiments, the acetate producing bacteria comprises a short chain fatty acid producing bacteria. In some embodiments, the acetate producing bacteria comprises Bifidobacteria or Allobaculum, or a combination thereof. In some embodiments, the IBD is ulcerative colitis. In some embodiments, the IBD is Crohn's disease. In some embodiments, the IBD is indeterminate colitis. In some embodiments, the method comprises administering to the subject a second agent. In some embodiments, the second agent comprises an inhibitor of Tumor necrosis factor (TNF)-like cytokine 1A (TL1A) activity or expression. In some embodiments, the inhibitor of TL1A activity or expression comprises an antibody or antigen-binding fragment thereof. In some embodiments, the antibody or antigen-binding fragment thereof binds to TL1A. In some embodiments, the antibody or antigen-binding fragment thereof disrupts binding between TL1A and Death receptor 3 (DR3). In some embodiments, the one or more samples comprises a stool sample, a blood sample, or a tissue biopsy, or any combination thereof. In some embodiments, the subject is predicted to develop the ileitis, intestinal fibrosis, or colitis based, at least in part, on an increase in expression of a gene expression product of TNF superfamily member 15 (TNFSF15) in the one or more samples obtained from the subject relative to a reference expression level obtained from a reference subject without the IBD. In some embodiments, wherein the subject is predicted to develop the ileitis, intestinal fibrosis, or colitis based, at least in part, on an increase in expression of a gene expression product of TNF superfamily member 15 (TNFSF15) and the increase in the amount of the acetate or the metabolite thereof in the one or more samples obtained from the subject relative to a reference levels obtained from a reference subject without the IBD. In some embodiments, the method comprises measuring the increase in expression of a gene expression product of TNFSF15 in the one or more samples obtained from the subject. In some embodiments, the method comprises measuring the increase in the amount of acetate or a metabolite thereof, or the amount of acetate producing bacteria in the one or more samples obtained from the subject. In some embodiments, the method comprises measuring the increase in the amount of 3-hydroxy-propionate in the one or more samples obtained from the subject. In some embodiments, the increase in the amount of acetate is measured by a process comprising performing an immunoassay on the one or more samples. In some embodiments, the immunoassay comprises a colorimetric readout. In some embodiments, the subject is predicted to develop the ileitis, intestinal fibrosis, or colitis based, at least in part, on detection of hyperplasia in a population of Paneth cells obtained from the one or more samples obtained from the subject. In some embodiments, the population of Paneth cells comprises a phenotype comprising D1, D3, D1234, or any combination thereof. In some embodiments, the hyperplasia in the population of the Paneth cells is detected by a process comprising lysozyme staining of ileal tissue sections derived from the one or more samples obtained from the subject. In some embodiments, the subject is predicted to develop the ileitis, intestinal fibrosis, or colitis based, at least in part, on an increase in a production of bile acids measured in the one or more samples obtained from the subject, relative to a reference level obtained from a reference subject without the IBD. In some embodiments, the method comprises measuring an increase in the production of bile acids relative to a reference level obtained from a reference subject without the IBD. In some embodiments, the bile acids comprise secondary bile acids. In some embodiments, the secondary bile acids comprise deoxycholic acid, lithocholic acid, or combinations thereof. In some embodiments, the subject is predicted to develop the ileitis, intestinal fibrosis, or colitis based, at least in part, the increase in the production of bile acids. In some embodiments, the subject is predicted to develop the ileitis, intestinal fibrosis, or colitis based, at least in part, an increase in production of interferon gamma (IFN-gamma) in the one or more samples obtained from the subject relative to a reference level obtained from a reference subject without the IBD. In some embodiments, the method comprises measuring the increase in the production of the IFN-gamma in the one or more samples relative to a reference level obtained from a reference subject without the IBD. In some embodiments, the subject is predicted to develop the ileitis, intestinal fibrosis, or colitis based, at least in part, an abnormal morphology of Paneth cells obtained from the one or more samples relative to a reference morphology obtained from a reference subject without the IBD. In some embodiments, the abnormal morphology comprises a distended or distorted endoplasmic reticulum, a distended or distorted mitochondria, or an increased vesiculation. In some embodiments, the abnormal morphology of the Paneth cells is measured using histological analysis of the one or more samples. In some embodiments, the abnormal morphology of the Paneth cells is measured using transmission electron microscopy. In some embodiments, the subject is predicted to develop the ileitis, the intestinal fibrosis, or the colitis based, at least in part, on an increase in expression of a gene expression product of tumor necrosis factor receptor superfamily member 25 (TNFRSF25) in the one or more samples obtained from the subject relative to the reference expression level obtained from the reference subject without IBD. In some embodiments, the method comprises measuring the increase in expression of the gene expression product of TNFRSF25 in the one or more samples relative to a reference expression level obtained from a reference subject without the IBD. In some embodiments, the gene expression product is mRNA encoding TL1A receptor Death receptor 3 (DR3). In some embodiments, the gene expression product is TL1A receptor Death receptor 3 (DR3). In some embodiments, the subject is predicted to develop the ileitis, the intestinal fibrosis, or the colitis based, at least in part, on a presence of one or more genetic risk variants of tumor necrosis factor receptor superfamily member 15 (TNFRSF15) in the one or more samples obtained from the subject. In some embodiments, the one or more genetic risk variants is one or more single nucleotide polymorphisms (SNPs). In some embodiments, the one or more SNPs comprise a haplotype at a locus in TNFSF15. In some embodiments, the one or more SNPs comprises rs3810936, rs6478 108, rs6478109, rs7848647, or rs7869487, or any combination thereof. In some embodiments, the one or more SNPs comprises a “C” at rs3810936, a “T” at rs6478 108, a “G” at rs6478 109, a “C” at rs7848647, or a “T” at rs7869487, or any combination thereof. In some embodiments, the one or more SNPs comprises a “T” at rs3810936, a “C” at rs6478108, a “A” at rs6478109, a “T” at rs7848647, a “C” at rs7869487, or any combination thereof. In some embodiments, the one or more SNPs comprises a “T” at rs3810936, a “C” at rs6478108, a “A” at rs6478109, a “T” at rs7848647, or a “T” at rs7869487, or any combination thereof. In some embodiments, the method comprises detecting the presence of the one or more genetic risk variants of INFSF15 in the one or more samples obtained from the subject. In some embodiments, the measuring the presence of the one or more genetic risk variants at TNFSF15 comprises performing a genotyping assay. In some embodiments, the subject is predicted to develop the ileitis, intestinal fibrosis, or colitis based, at least in part, on a reduction of protective short-chain fatty acids measured in the one or more samples relative to a reference level obtained from a reference subject without the IBD. In some embodiments, the method comprises measuring the reduction in production of protective short-chain fatty acids in the one or more samples obtained from the subject. In some embodiments, the protective short chain fatty acids comprise butyrate, or propionate, or a combination thereof.

In certain aspects, disclosed herein is a method of predicting a high likelihood that a subject with inflammatory bowel disease (IBD) will develop ileitis, fibrotic disease, colitis, or ulcerative colitis, the method comprising: measuring an increase in an amount of acetate or a metabolite thereof, or an amount of acetate producing bacteria in one or more samples obtained from the subject, wherein the increase in the amount of acetate or the metabolite thereof, or the acetate producing bacteria is relative to a reference level obtained from a reference subject without the IBD; and predicting the high likelihood that the subject with IBD will develop the ileitis, intestinal fibrosis, or colitis based, at least in part, on the increase in the amount of acetate or the metabolite thereof, or the acetate producing bacteria measured in (a). In some embodiments, measuring the increase in the amount of acetate in the sample comprises performing an immunoassay. In some embodiments, the immunoassay comprises a colorimetric readout. In some embodiments, the acetate producing bacteria comprises Bifidobacteria or Allobaculum, or a combination thereof. In some embodiments, the acetate producing bacteria also produces 3-hydroxy-propionate. In some embodiments, the method comprises detecting hyperplasia in a population of Paneth cells obtained from the one or more samples obtained from the subject. In some embodiments, the population of Paneth cells comprises a phenotype comprising D1, D3, D1234, or any combination thereof. In some embodiments, the hyperplasia in the population of the Paneth cells is detected by a process comprising lysozyme staining of ileal tissue sections derived from the one or more samples obtained from the subject. In some embodiments, the predicting of (b) further comprises predicting the high likelihood that the subject with IBD will develop the ileitis, the intestinal fibrosis, or the colitis based, at least in part, on the hyperplasia in the population of the Panel cells. In some embodiments, the IBD is ulcerative colitis. In some embodiments, the IBD is Crohn's disease. In some embodiments, the method comprises measuring a reduction in production of protective short-chain fatty acids relative to a reference level obtained from a reference subject without the IBD. In some embodiments, the protective short chain fatty acids comprise butyrate, or propionate. In some embodiments, the predicting of (b) further comprises predicting the likelihood that the subject with IBD will develop the ileitis, intestinal fibrosis, or colitis based, at least in part, on the reduction of the protective short-chain fatty acids measured in the one or more samples obtained from the subject. In some embodiments, the protective short-chain fatty acids measured in the one or more samples obtained from the subject comprise butyrate, propionate, or any combination thereof. In some embodiments, the one or more samples comprises a stool sample, a blood sample, or a tissue biopsy sample, or any combination thereof. In some embodiments, the measuring the reduction in the production of the protective short-chain fatty acids is performed by a process comprising performing quantitative polymerase chain reaction (qPCR) of a bacterial gene coding butyryl-CoA:acetate CoA-transferase. In some embodiments, the measuring the reduction in the production of the protective short-chain fatty acids is performed by a process comprising performing an immunoassay to measure an increase in the short-chain fatty acids directly. In some embodiments, the method comprises measuring an increase in the production of bile acids in the one or more samples obtained from the subject relative to a reference level obtained from a reference subject without the IBD. In some embodiments, the bile acids comprise secondary bile acids. In some embodiments, the secondary bile acids comprise deoxycholic acid, lithocholic acid, or combinations thereof. In some embodiments, the predicting of (b) further comprises predicting the high likelihood that the subject with IBD will develop the ileitis, intestinal fibrosis, or colitis based, at least in part, on the increase of the secondary bile acids, deoxycholic acid, lithocholic acid, or combinations thereof. In some embodiments, the method comprises measuring an increase in production of IFN-gamma relative to a reference level obtained from a reference subject without the IBD. In some embodiments, the predicting of (b) further comprises predicting the high likelihood that the subject with IBD will develop the ileitis, intestinal fibrosis, or colitis based, at least in part, on the increase in the production of IFN-gamma. In some embodiments, the method comprises measuring an abnormal morphology of subject Paneth cells in the one or more samples obtained from the subject relative to a reference morphology obtained from a reference subject without the IBD. In some embodiments, the abnormal morphology comprises a distended or distorted endoplasmic reticulum, a distended or distorted mitochondria, or an increased vesiculation. In some embodiments, the predicting of (b) further comprises predicting the high likelihood that the subject with IBD will develop the ileitis, intestinal fibrosis, or colitis based, at least in part, on the abnormal morphology of Paneth cells. In some embodiments, the method comprises measuring an increase in expression of a gene expression product of TNFSF25 in the one or more samples obtained from the subject relative to a reference expression level obtained from a reference subject without the IBD. In some embodiments, the predicting of (b) further comprises predicting the high likelihood that the subject with IBD will develop the ileitis, intestinal fibrosis, or colitis based, at least in part, on the increase in the expression of gene expression product of TNFSF25. In some embodiments, the gene expression product of TNFSF25 is mRNA encoding DR3. In some embodiments, the gene expression product of TNFSF25 is DR3. In some embodiments, the method comprises measuring an increase in expression of a gene expression product of TNFSF15 in the one or more samples obtained from the subject relative to a reference expression level obtained from a reference subject without the IBD. In some embodiments, the predicting of (b) further comprises predicting the likelihood that the subject with IBD will develop the ileitis, intestinal fibrosis, or colitis based, at least in part, on the increase in the expression of the gene expression product of TNFSF15. In some embodiments, the predicting of (b) further comprises predicting the likelihood that the subject with IBD will develop the ileitis, intestinal fibrosis, or colitis based, at least in part, on the increase in the expression of the gene expression product of TNFSF15, and the increase in the amount of acetate or the metabolite thereof. In some embodiments, the increase in the expression of the gene expression product of TNFSF15 is measured by a process comprising: (a) stimulating peripheral blood mononuclear cells (PBMCs) obtained from the one or more samples obtained from the subject with immune complex under conditions sufficient for the PBMCs to express the gene expression product; (b) measuring the expression of the gene expression product; and (c) comparing the expression of the gene expression product with the reference expression level. In some embodiments, the increase in the expression of the gene expression product of TNFSF15 is measured by a process comprising single molecule protein detection. In some embodiments, the wherein the gene expression product is mRNA encoding TL1A. In some embodiments, the wherein the gene expression product is TL1A. In some embodiments, the method comprises detecting a presence of one or more genetic risk variants at TNF superfamily member 15 (TNFSF15) in the one or more samples obtained from the subject. In some embodiments, the one or more genetic risk variants is one or more single nucleotide polymorphisms (SNPs). In some embodiments, the one or more SNPs belong a haplotype at a locus in TNFSF15. In some embodiments, the one or more SNPs comprises rs3810936, rs6478108, rs6478109, rs7848647, or rs7869487, or any combination thereof. In some embodiments, the one or more SNPs comprises a “C” at rs3810936, a “T” at rs6478 108, a “G” at rs6478 109, a “C” at rs7848647, or a “T” at rs7869487, or any combination thereof. In some embodiments, the one or more SNPs comprises a “T” at rs3810936, a “C” at rs6478108, a “A” at rs6478109, a “T” at rs7848647, a “C” at rs7869487, or any combination thereof. In some embodiments, the one or more SNPs comprises a “T” at rs3810936, a “C” at rs6478108, a “A” at rs6478109, a “T” at rs7848647, or a “T” at rs7869487, or any combination thereof. In some embodiments, the measuring the presence of the one or more genetic risk variants at TNFSF15 comprises performing a genotyping assay.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-1I show graphs and figures illustrating the association between TL1A-overexpression and abnormal Paneth cell morphology in mice and Crohn's disease (CD) patients. FIG. 1A shows the number of Paneth cells per crypt in wild type (WT) and Tl1a-tg mice at indicated time-points. FIG. 1B shows the inflammation score in WT and Tl1a-tg mice at indicated time-points. FIG. 1C shows the lysozyme staining in 6-month-old WT and Tl1a-tg mice. FIGS. 1D-1F show the percentage of total Paneth cells with D0, D1, D2, and D3 features in WT and Tl1a-tg mice at indicated mice ages (e.g., 4-6 weeks, 2-4 months, and 6 months). FIG. 1G shows the transmission electron microscopy images of WT and Tl1a-tg Paneth cells. FIG. 1H shows mucosal TL1A mRNA expression compared to the percentage of abnormal Paneth cells in CD patients. FIG. 1I shows DR3 expressed on murine ileal Paneth cells.

FIGS. 2A-2F show graphs illustrating that DR3 signaling regulates the development and function of ileal Paneth cells in a cell-intrinsic manner. FIGS. 2A-2C show Paneth cell Phenotype in WT, Tl1a-tg, Dr3^{ΔPC}, and Tl1a-tg Dr3^{ΔPC} mice at 2 to 4 months of age. FIG. 2A shows the percentage of total Paneth cells with D0, D1, D2, and D3 features. FIG. 2B shows the number of Paneth cells per crypt in WT, Tl1a-tg, Dr3^{ΔPC}, and Tl1a-tg Dr3^{ΔPC} mice. FIG. 2C shows the inflammation score in WT, Tl1a-tg, Dr3^{ΔPC}, and Tl1a-tg Dr3^{ΔPC} mice. FIGS. 2D-2F show the Paneth cell Phenotype in WT, Tl1a-tg, Dr3^{ΔPC}, and Tl1a-tg Dr3^{ΔPC} mice at 6 months of age. FIG. 2D shows the percentage of total Paneth cells with D0, D1, D2, and D3 features. FIG. 2E shows the number of Paneth cells per crypt in WT, Tl1a-tg, Dr3^{ΔPC}, and Tl1a-tg Dr3^{ΔPC} mice. FIG. 2F shows the inflammation score in WT, Tl1a-tg, Dr3^{ΔPC}, and Tl1a-tg Dr3^{ΔPC} mice.

FIG. 3 shows the expression of human DR3 in non-inflamed small intestinal tissue sections from a CD patient.

FIGS. 4A-4C show graphs and charts illustrating that TL1A-overexpression leads to transcriptional changes in Paneth cells preceding small intestinal inflammation and is associated with abnormal Paneth cell granules and ER stress. FIG. 4A shows a heat map displaying RNA sequencing data of 59 genes differentially expressed. FIG. 4B shows the enrichment of pathways from genes differentially expressed in WT and Tl1a-tg Paneth cells. FIG. 4C shows the quantification of TUNEL⁺ cells in ileal crypts of 2-month-old and 6-month-old WT and Tl1a-tg mice.

FIGS. 5A-5L show graphs illustrating that intestinal microbiota are required for Paneth cell granule maturation and ileal inflammation. FIGS. 5A-5D show Paneth cell phenotypes in germ-free (GF) WT and Tl1a-tg mice. FIG. 5A shows the percentage of total Paneth cells with D0, D1, D2, and D3 features for mice 4-5 months of age. FIG. 5B shows the percentage of total Paneth cells with D0, D1, D2, and D3 features for mice at 10 months of age.

FIG. 5C shows the numbers of Paneth cells per crypt for WT and Tl1a-tg mice. FIG. 5D shows the inflammation score for WT and Tl1a-tg mice. FIGS. 5E-5G show the Paneth cell phenotypes in GF WT mice reconstituted with microbiota from SPF mice at 2-4 months of age. FIG. 5E shows the percentage of total Paneth cells with D0, D1, D2, and D3 features for WT and Tl1a-tg mice at 10 months of age. FIG. 5F shows the number of Paneth cells per Crypt for WT and Tl1a-tg mice. FIG. 5G shows the inflammation score for WT and tl1a-tg mice. FIG. 5H shows the Log 2 fold change in abundance of various bacterial strains in Germ-free TL1a-tg and WT mice reconstituted with SPF microbiota. FIG. 5I shows the Log 2 fold change in abundance of various bacterial strains in SPF TL1a-tg and WT mice. FIG. 5J shows a sparse partial least squares discriminant analysis plot depicting metabolome profiles in the ileum of SPF WT or Tl1a-tg mice. FIG. 5K shows the amount of acetate in WT mice and Tl1a-tg mice. FIG. 5L shows the amount of 3-hydroxy propionate in WT mice and Tl1a-tg mice.

FIGS. 6A-6E show images and graphs illustrating that the microbial metabolite acetate promotes ileal inflammation in GF WT mice. FIG. 6A shows the experimental setup for acetate supplementation of GF WT mice. FIG. 6B shows the ileum histoscores of WT H2O, WT Ac, Tl1a-tg H2O, and Tl1a-tg Ac mice. FIG. 6C shows representative hematoxylin and eosin (H&E) images of WT H2O, WT Ac, Tl1a-tg H2O, and tl1a-tg Ac mice. FIG. 6D shows IFN-γ secretion of MLN of water or acetate treated GF WT or Tl1a-tg mice. FIG. 6E shows the percentage of total Paneth cells with D0, D1, D2, and D3 features of WT acetate or tl1a-tg acetate mice.

FIGS. 7A-7C show graphs and images illustrating that the microbial metabolite acetate promotes ileal inflammation in SPF mice. FIG. 7A shows the experimental setup for acetate supplementation of SPF mice. FIG. 7B shows the histoscores from the experimental setup in FIG. 7A for WT H2O, WT Ac, Tl1a-tg H2O, and Tl1a-tg Ac mice. FIG. 7C shows representative H&E images.

FIGS. 8A-8B show graphs illustrating that SPF TI1a-tg mice have a higher abundance of short-chain-fatty-acid producing bacteria and the microbial metabolites acetate and formic acid when compared to WT mice. FIG. 8A shows graphs illustrating the acetic acid levels in the cecum. FIG. 8B shows a graph illustrating the formic acid levels in the cecum of WT and Tl1a-Tg mice.

FIG. 9 shows a computer system that is programmed or otherwise configured to implement methods provided herein.

FIG. 10 shows a graph illustrating that acetate induces cecal collagen deposition (fibrosis) in Germ-free IBD susceptible mice.

FIG. 11 shows a workflow according to an embodiment of the present disclosure for processing a biological sample obtained from a subject.

FIG. 12 shows a computer-implemented workflow according to an embodiment of the present disclosure for generating an electronic report to a user, such as a physician, comprising a genetic risk score of a subject based on an analysis of genotype data from the subject.

### DETAILED DESCRIPTION

Provided herein are methods, systems, and kits for identifying a subject who may develop ileitis, intestinal fibrosis, or colitis based on a presence or a level of one or more biomarkers present in a sample obtained from the subject. The subject may be a patient, who may be diagnosed with an inflammatory disease, a fibrostenotic disease, or a fibrotic disease, such as inflammatory bowel disease (IBD). The inflammatory bowel disease may comprise Crohn's disease or ulcerative colitis. The subject may not be a patient but may be suspected of having or developing the ileitis, intestinal fibrosis, or colitis. In some embodiments, the one or more biomarkers comprises a genotype (e.g., one or more genetic risk variants) at TNF superfamily member 15 (TNFSF15); an analyte (e.g., acetate); a metabolite (e.g., 3-hydroxy-propionate); a gene expression product expressed from TNFSF15, tumor necrosis factor receptor superfamily member 25 (TNFRSF25), or both; an abnormal morphology, hyperplasia, or both of Paneth cells; bile acids; one or more acetate producing bacteria (e.g., Bifidobacteria or Allobaculum); protective short-chain fatty acids (e.g., butyrate); interferon gamm (IFN-gamma); or any combination thereof. The one or more biomarkers may, in some cases, be useful for characterizing the develop ileitis, intestinal fibrosis, or colitis, as mediated by acetate. The subject, in some embodiments, is treated by administering a therapeutic agent to the subject, provided the presence or the level of one or more biomarkers is detected in a sample obtained from the subject. In some embodiments, the therapeutic agent comprises an inhibitor of acetate-producing bacteria. In some embodiments, the therapeutic agent comprises an inhibitor of Tumor necrosis factor (TNF)-like cytokine 1A (TL1A), such as for example, an anti-TL1A antibody or antigen-binding fragment thereof. In some embodiments, the level of the one or more biomarkers is higher or lower than a reference level obtained from a reference subject. In some embodiments, the reference subject does not have the IBD. In some embodiments, the reference subject does not have the ileitis, intestinal fibrosis, or colitis. In some embodiments, the methods disclosed herein provide for identifying the subject as being suitable for treatment with the therapeutic agent based on the one or more biomarkers detected in a sample obtained from the subject prior to administering the therapeutic agent to the subject.

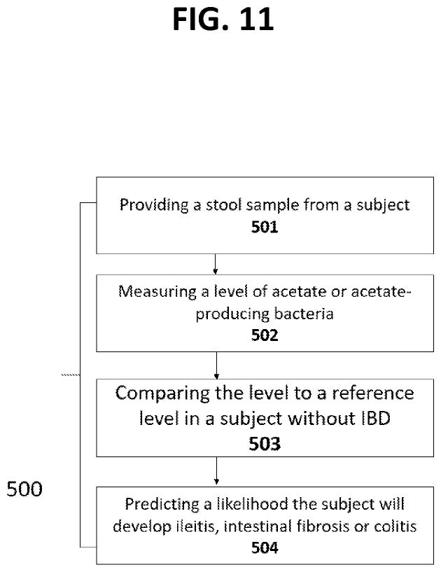

Referring to FIG. 11, the methods, systems and kits of the present disclosure involve, in some embodiments, the steps of providing a stool sample from a subject 501 (although other biological samples and methods may be substituted), assaying the optionally processed sample to detect a level of acetate of an acetate producing bacteria in the subject 502, comparing the level to a reference level in a subject without IBD 503; and predicting a likelihood the subject will develop ileitis, intestinal fibrosis or colitis. In some embodiments, the methods, systems and kits involve assaying the stool sample or another sample (e.g., a blood sample) to measure or detect another biomarker, such as a genotype (e.g., one or more genetic risk variants) at TNFSF15; an analyte (e.g., acetate); a metabolite (e.g., 3-hydroxy-propionate); a gene expression product expressed from TNFSF15, TNFRSF25, or both (e.g., TL1A, DR3, mRNA encoding TL1A or DR3, or a combination thereof); an abnormal morphology, hyperplasia, or both of Paneth cells; bile acids; one or more acetate producing bacteria (e.g., Bifidobacteria or Allobaculum); protective short-chain fatty acids (e.g., butyrate); interferon gamm (IFN-gamma); or any combination thereof. In some embodiments, the methods, systems and kits involve comparing the level of the biomarker to a reference level of the biomarker obtained from a reference subject, such as for example a subject without the ileitis, intestinal fibrosis, or colitis. In some embodiments, the methods, systems, and kits involve predicting a likelihood the subject will develop ileitis, intestinal fibrosis or colitis, based at least in part on the level or the presence of one or more of the biomarkers disclosed herein.

Also provided are methods, systems, and kits for detecting one or more biomarkers disclosed herein in one or more samples obtained from a subject. The biomarkers (e.g., genotypes, mRNA) described herein are detected using suitable genotyping devices (e.g., array, sequencing, qPCR, TaqMan PCR). The biomarkers disclosed herein may be detected using suitable immunoassays, such as enzyme immunoassay (EIA), radioimmunoassay (RIA), fluoroimmunoassay (FIA), chemiluminescent immunoassay (CLIA) or counting immunoassay (CIA). In some instances, the one or more samples is obtained from the subject or patient indirectly or directly. In some instances, the one or more samples may be obtained by the subject. In other instances, the one or more samples may be obtained by a healthcare professional, such as a nurse or physician. The one or more samples may be derived from virtually any biological fluid or tissue containing genetic information, proteins, pathogens or analytes, such as blood.

In certain embodiments, described herein are methods, systems and kits for evaluating an effect of a treatment described herein. In some instances, the treatment comprises administration with a therapeutic agent and optionally, one or more additional therapeutic agents. In some instances, the treatment is monitored by evaluating the levels of acetate or acetate-producing bacteria in the subject prior to and/or after administration of a therapeutic agent.

In some embodiments, the methods, systems and kits characterize the treatment of a subject. In some embodiments, the methods, systems and kits monitor treatment. In some embodiments, the methods, systems and kits select a subject for treatment. In some embodiments, the method, systems and kits reduce the levels of acetate or acetate-producing bacteria in a subject. In some embodiments, the subject has an IBD.

### Biomarker Detection

Disclosed herein are methods, systems, and kits for detecting one or more biomarkers in a sample obtained from a subject. In some embodiments, the one or more biomarkers are useful for predicting a high likelihood that a subject will develop ileitis, intestinal fibrosis, or colitis. In some embodiments, the subject has inflammatory bowel disease (IBD), such as Crohn's disease or ulcerative colitis. In some embodiments, methods disclosed herein comprise: (a) measuring a variation in an amount of one or more biomarkers in one or more samples obtained from the subject, wherein the variation in the amount of the one or more biomarkers is relative to a reference level obtained from a reference subject; and (b) predicting the high likelihood that the subject will develop the ileitis, intestinal fibrosis, or colitis based, at least in part, on the variation in the one or more biomarkers measured in (a). For example, in the case of genetic variants, the variation in the amount of a genetic variant disclosed herein may be a presence or an absence of one or more single nucleotide polymorphisms (SNPs) disclosed herein. In another example, the variation in the amount of an analyte, an enzyme, a cell type, a cell morphology, cell size, a metabolite, a polypeptide or ribonucleic acid (RNA), a microbial antigen or pathogen may be an increase or a decrease relative to the reference sample. The reference sample may be from a reference subject, or an index derived from a plurality of reference subject.

In some embodiments, the one or more biomarkers comprises a genetic variant, an analyte, an enzyme, a cell type, a cell morphology, cell size, a cell number, a metabolite, a polypeptide or ribonucleic acid (RNA), a microbial antigen or pathogen, or any combination thereof. In some embodiments, the genetic variant comprises one or more SNPs at TNFRSF15. In some embodiments, the one or more SNPs are at one or more of rs3810936, rs6478 108, rs6478109, rs7848647, or rs7869487. In some embodiments, the analyte comprises a short-chain fatty acid. In some embodiments, the short-chain fatty acid comprises acetate, butyrate, or propionate, or a combination thereof. In some embodiments, the cell type comprises a Paneth cell. In some embodiments, the Paneth cell is characterized by a phenotype associated with the ileitis, intestinal fibrosis or colitis. In some embodiments, the phenotype comprises abnormal Paneth cell granules D1, D3, D1234, or any combination thereof. In some embodiments, the cell morphology comprises a distended or distorted endoplasmic reticulum, a distended or distorted mitochondria, or an increased vesiculation, or a combination thereof. In some embodiments, the cell number is an increase in number (e.g., hyperplasia) in a population of Paneth cells. In some embodiments, the metabolite comprises 3-hydroxy-propionate. In some embodiments, the polypeptide comprises TL1A, DR3, IFN-gamma, or a combination thereof. In some embodiments, the RNA comprises messenger RNA (mRNA). In some embodiments, the mRNA encodes TL1A, DR3, IFN-gamma or a combination thereof. In some embodiments, the TL1A is soluble TL1A. In some embodiments, the DR3 is membrane DR3. In some embodiments, the microbial antigen comprises an acetate producing bacteria, such as for example, Bifidobacteria or Allobaculum. In some embodiments, the subject is predicted to develop the ileitis, intestinal fibrosis, or colitis based, at least in part, on an increase in a genetic variant, an analyte, an enzyme, a cell type, a cell morphology, cell size, a cell number, a metabolite, a polypeptide or ribonucleic acid (RNA), a microbial antigen or pathogen, or any combination thereof disclosed herein.

In some embodiments, the subject is predicted to develop the ileitis, intestinal fibrosis, or colitis based, at least in part, on an increase in expression of a gene expression product of TNFSF15 in the one or more samples obtained from the subject relative to a reference expression level obtained from a reference subject. In some embodiments, the subject is predicted to develop the ileitis, intestinal fibrosis, or colitis based, at least in part, on an increase in expression of a gene expression product of TNFSF25 in the one or more samples obtained from the subject relative to a reference expression level obtained from a reference subject. In some embodiments, the gene expression product of TNFSF15 is TL1A or mRNA encoding TL1A. In some embodiments, the gene expression product of TNFSF25 is DR3 or mRNA encoding DR3. In some embodiments, a presence of one or more genetic risk variants disclosed herein may be detected as a surrogate for the increase in the gene expression product of TNFSF15. In some embodiments, the subject is predicted to develop the ileitis, intestinal fibrosis, or colitis based, at least in part, on an increase in expression of a gene expression product of TNFSF15 and an increase in the amount of the acetate or the metabolite thereof in the one or more samples obtained from the subject relative to a reference levels obtained from a reference subject. In some embodiments, the methods further comprise measuring the increase in expression of a gene expression product of TNFSF15 in the one or more samples obtained from the subject. In some embodiments, the methods further comprise measuring the increase in the amount of acetate or the metabolite thereof, or the amount of acetate producing bacteria in the one or more samples obtained from the subject. In some embodiments, the reference subject does not have IBD. In some embodiments, the reference subject does not have the ileitis, intestinal fibrosis or colitis.

The subject disclosed herein can be a mammal, such as for example a mouse, rat, guinea pig, rabbit, non-human primate, human or farm animal. In some instances, the subject is human. In some instances, the subject is suffering from a symptom related to a disease or condition disclosed herein (e.g., abdominal pain, cramping, diarrhea, rectal bleeding, fever, weight loss, fatigue, loss of appetite, dehydration, and malnutrition, anemia, or ulcers).

In some embodiments, the subject has a disease or a condition disclosed herein. In some embodiments, the disease or the condition is an inflammatory bowel disease (IBD), such as Crohn's disease (CD) or ulcerative colitis (UC). In some embodiments, the disease or condition is ileitis. As used herein, “ileitis” comprises an inflammation of the ileum. In some embodiments, the disease or condition is colitis. As used herein, “colitis” comprises an inflammation of the colon. In some embodiments, the IBD is indeterminate colitis. A subject may suffer from fibrosis, fibrostenosis, or a fibrotic disease, either isolated or in combination with an inflammatory disease. In some cases, the CD is severe CD. The severe CD may result from inflammation that has led to the formation of scar tissue in the intestinal wall (fibrostenosis) and/or swelling. In some cases, the severe CD is characterized by the presence of fibrotic and/or inflammatory strictures. The strictures may be determined by computed tomography enterography (CTE), and magnetic resonance imaging enterography (MRE). The disease or condition may be characterized as refractory, which in some cases, means the disease is resistant to a first-line treatment or a non-curative treatment. In some embodiments, the first-line therapy comprises an inhibitor of tumor necrosis factor (TNF). In some embodiments, the inhibitor of TNF is an anti-TNF antibody or antigen binding fragment thereof. In some embodiments, the anti-TNF antibody comprises infliximab, adalimumab, etanercept, golimumab, or certolizumab. Non-limiting examples of non-curative treatments include corticoid steroids (e.g., budesonide), thalidomide, and cyclophosphamide. In some embodiments, the first-line therapy comprises an inhibitor of α4-β7 integrin, such as vedolizumab. In some embodiments, the first-line therapy comprises an inhibitor of the p40 subunit of interleukin 12 (IL-12), such as ustekinumab.

The term “sample” as used here generally refers to a biological sample. In some embodiments, methods comprise obtaining a sample from a subject described herein. In some instances, the subject may be human. In some embodiments, the sample is obtained from a subject having a disease or condition disclosed herein. In some cases, the sample is obtained from tissue biopsy, blood, serum, plasma, sweat, hair, tears, urine, stool, and other suitable sources. Techniques for obtaining samples from a subject include, for example, obtaining samples by a mouth swab or a mouth wash, drawing blood, and obtaining a biopsy. In some cases, the sample is obtained from a biopsy, e.g., from the intestinal track of the subject. In some embodiments, the sample is a blood sample. In some embodiments, the sample is a stool sample. In some embodiments, the sample is a tissue biopsy. In some embodiments, the sample is an intestinal biopsy. Isolating components of fluid or tissue samples (e.g., cells or RNA or DNA) may be accomplished using a variety of suitable techniques, such as those disclosed in Nichols Z E, et al., Sample Preparation and Diagnostic Methods for a Variety of Settings: A Comprehensive Review. Molecules. 2021 Sep. 18; 26(18):5666, which is hereby incorporated by reference in its entirety. After the sample is obtained, it may be further processed to enrich for or purify genomic material.

Described herein, in some embodiments, are methods, systems and kits for detecting a metabolite. In some embodiments, the methods described herein comprise detection of a metabolite. In some embodiments, the methods comprise detecting, a bile acid, interferon-gamma, or short-chain fatty acids. In some embodiments, the short-chain fatty acid comprises acetate, butyrate, or propionate, or any combination thereof. In some embodiments, the methods further comprising measuring the increase in the metabolite. In some embodiments, the level of the metabolite is increased. In some embodiments, the level of the metabolite is decreased. In some embodiments, the production of the metabolite is increased. In some embodiments, the production of the metabolite is decreased. In some embodiments, the level or production of the metabolite is increased relative to a reference level. In some embodiments, the level or production of the metabolite is decreased relative to a reference level. The reference level may be obtained from a reference subject or a population of reference subjects. In some embodiments, the reference subject does not have IBD. In some embodiments, the reference subject does not have CD. In some embodiments, the reference subject does not have the ileitis, intestinal fibrosis, or colitis. In some embodiments, the reference subject does not have the ileitis. In some embodiments, the reference subject does not have the intestinal fibrosis. In some embodiments, the reference subject does not have the colitis.

In some embodiments, the metabolite is acetate or a metabolite thereof. In some embodiments, the amount of acetate is measured by a process comprising performing an immunoassay on the one or more samples. In some embodiments, the immunoassay comprises a colorimetric readout. In some embodiments, the metabolite is 3-hydroxy-propionate.

In some embodiments, the secondary bile acid comprises deoxycholic acid, lithocholic acid, or a combination thereof. In some embodiments, the bile acid is measured by a process comprising performing an immunoassay on the one or more samples. In some embodiments, the immunoassay comprises a colorimetric readout.

In some embodiments, the protein is interferon-gamma (IFN-gamma). In some embodiments, the amount of acetate is measured by a process comprising performing an immunoassay on the one or more samples. In some embodiments, the immunoassay comprises a colorimetric readout.

In some embodiments, the metabolite is a protective short-chain fatty acid. In some embodiments, butyrate, or propionate, or a combination thereof.

Described herein, in some embodiments, are methods, systems and kits for detecting a short-chain fatty acid producing bacteria. In some embodiments, the short-chain fatty acid producing bacteria is an acetate-producing bacteria. In some embodiments, the methods described herein comprise detecting an acetate producing bacteria. In some embodiments, the acetate producing bacteria comprises Bifidobacteria, Allobaculum, or a combination thereof. In some embodiments, the acetate producing bacteria comprises Bifidobacteria. In some embodiments, the acetate producing bacteria comprises Allobaculum.

Described herein, in some embodiments, are methods, systems and kits for detecting a phenotype of one or more cells derived from the sample obtained from the subject. In some embodiments, the one or more cells comprises an immune cell, such as neutrophils, eosinophils, basophils, mast cells, monocytes, macrophages, dendritic cells, natural killer cells, or lymphocytes, In some embodiments, the one or more cells comprises a cell in the intestinal epithelium, such as enterocytes, Paneth cells, goblet cells, or neuroendocrine cells. In some embodiments, the one or more cells comprises a Paneth cell or a population of Paneth cells. In some embodiments, the sample is an ileal sample. As used herein, “Paneth cells” comprise secretory cells located in the small intestinal epithelium. In some cases, Paneth cells secrete antimicrobial peptides.

In some embodiments, the phenotype is hyperplasia. As defined herein, “hyperplasia” refers to an increase in the number of Paneth cells. In some embodiments, the population of Paneth cells comprises a phenotype comprising D1, D3, D1234, or any combination thereof. In some embodiments, the hyperplasia in the population of the Paneth cells is detected by a process comprising lysozyme staining of the one or more samples obtained from the subject. In some embodiments, hyperplasia may be measured compared to a reference number of Paneth cells in a sample derived from a subject with out IBD.

Described herein, in some embodiments, are methods, systems and kits for detecting an abnormal morphology of one or more cells derived from the sample obtained from the subject. In some embodiments, the one or more cells comprises an immune cell, such as neutrophils, eosinophils, basophils, mast cells, monocytes, macrophages, dendritic cells, natural killer cells, or lymphocytes, In some embodiments, the one or more cells comprises a cell in the intestinal epithelium, such as enterocytes, Paneth cells, goblet cells, or neuroendocrine cells. In some embodiments, the one or more cells comprises a Paneth cell or a population of Paneth cells. In some embodiments, the abnormal morphology is compared to a reference morphology derived from a subject. In some embodiments, the reference subject does not have IBD. In some embodiments, the reference subject does not have the ileitis, intestinal fibrosis or colitis. In some embodiments, the abnormal morphology comprises a distended or distorted endoplasmic reticulum, a distended or distorted mitochondria, or an increased vesiculation. In some embodiments, the abnormal morphology comprises a distended or distorted endoplasmic reticulum. In some embodiments, the abnormal morphology comprises a distended or distorted mitochondria. In some embodiments, the abnormal morphology comprises an increased vesiculation. In some embodiments, the abnormal morphology is measured using a histological analysis. In some embodiments, the abnormal morphology is measured using H&E staining. In some embodiments, the abnormal morphology is measured using microscopy. In some embodiments, the abnormal morphology of the Paneth cells is measured using transmission electron microscopy. In some embodiments, the subject is predicted to develop the ileitis, intestinal fibrosis, or colitis based, at least in part, an abnormal morphology of Paneth cells.

Disclosed herein, in some embodiments, are systems, methods and kits for detecting one or more genotypes. In some embodiments, the genotypes described herein are detected using suitable genotyping devices (e.g., array, sequencing). In some instances, a sample is obtained from the subject or patient indirectly or directly. In some instances, the sample may be obtained by the subject. In other instances, the sample may be obtained by a healthcare professional, such as a nurse or physician. The sample may be derived from virtually any biological fluid or tissue containing genetic information, such as blood.

In certain aspects, the subject is predicted to develop the ileitis, the intestinal fibrosis, or the colitis based, at least in part, on a presence of one or more genetic risk variants of TNFRSF15 in the one or more samples obtained from the subject, wherein the one or more genetic risk variants is one or more single nucleotide polymorphisms (SNPs). In some embodiments, the one or more SNPs are at rs3810936, rs6478108, rs6478109, rs7848647, or rs7869487. In some embodiments, a position of one or more variant nucleotides at rs3810936, rs6478108, rs6478 109, rs7848647, or rs7869487 are indicated with a non-nucleobase letter (e.g., R, N, S) in National Library of Medicine (NCBI) dsSNP database, which flanking sequences are hereby incorporated by reference in their entirety. In some embodiments the variant nucleotide of interest comprises a “C” at rs3810936, a “T” at rs6478108, a “G” at rs6478109, a “C” at rs7848647, or a “T” at rs7869487, or any combination thereof. In some embodiments, the variant nucleotide of interest comprises a “T” at rs3810936, a “C” at rs6478108, a “A” at rs6478109, a “T” at rs7848647, a “C” at rs7869487, or any combination thereof. In some embodiments, the variant nucleotide of interest comprises a “T” at rs3810936, a “C” at rs6478108, a “A” at rs6478109, a “T” at rs7848647, or a “T” at rs7869487, or any combination thereof.

Disclosed herein are genotypes that may be detected in a sample obtained from a subject by analyzing the genetic material in the sample. In some instances, the subject may be human. In some embodiments, the genetic material is obtained from a subject having a disease or condition disclosed herein. In some cases, the genetic material is obtained from blood, serum, plasma, sweat, hair, tears, urine, stool, and other techniques known by one of skill in the art. In some cases, the genetic material is obtained from a biopsy, e.g., from the intestinal track of the subject.

The genotypes of the present disclosure comprise genetic material that is deoxyribonucleic acid (DNA). In some instances, the genotype comprises a denatured DNA molecule or fragment thereof. In some instances, the genotype comprises DNA selected from:
genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented.

Methods disclosed herein for detecting a genotype in a sample from a subject comprise analyzing the genetic material in the sample to detect at least one of a presence, an absence, and a quantity of a nucleic acid sequence encompassing the genotype of interest. In some embodiments, the sample is assayed to measure a presence, absence, or quantity of at least one, two, or three polymorphisms. In some embodiments, the sample is assayed to measure a presence, absence, or quantity of at least four polymorphisms. In some embodiments, the sample is assayed to measure a presence, absence, or quantity of at least five polymorphisms. In some embodiments, at least one, two, or three genotypes are detected, using the methods described herein.

In some cases, the nucleic acid sequence comprises DNA. In some instances, the nucleic acid sequence comprises a denatured DNA molecule or fragment thereof. In some instances, the nucleic acid sequence comprises DNA selected from: genomic DNA, viral DNA, mitochondrial DNA, plasmid DNA, amplified DNA, circular DNA, circulating DNA, cell-free DNA, or exosomal DNA. In some instances, the DNA is single-stranded DNA (ssDNA), double-stranded DNA, denaturing double-stranded DNA, synthetic DNA, and combinations thereof. The circular DNA may be cleaved or fragmented. In some instances, the nucleic acid sequence comprises RNA. In some instances, the nucleic acid sequence comprises fragmented RNA. In some instances, the nucleic acid sequence comprises partially degraded RNA. In some instances, the nucleic acid sequence comprises a microRNA or portion thereof. In some instances, the nucleic acid sequence comprises an RNA molecule or a fragmented RNA molecule (RNA fragments) selected from: a microRNA (miRNA), a pre-miRNA, a pri-miRNA, a mRNA, a pre-mRNA, a viral RNA, a viroid RNA, a virusoid RNA, circular RNA (circRNA), a ribosomal RNA (rRNA), a transfer RNA (tRNA), a pre-tRNA, a long non-coding RNA (lncRNA), a small nuclear RNA (snRNA), a circulating RNA, a cell-free RNA, an exosomal RNA, a vector-expressed RNA, an RNA transcript, a synthetic RNA, and combinations thereof.

Nucleic acid-based detection techniques that may be useful for the methods herein include quantitative polymerase chain reaction (qPCR), gel electrophoresis, immunochemistry, in situ hybridization such as fluorescent in situ hybridization (FISH), cytochemistry, and next generation sequencing. In some embodiments, the methods involve TaqMan™ qPCR, which involves a nucleic acid amplification reaction with a specific primer pair, and hybridization of the amplified nucleic acids with a hydrolysable probe specific to a target nucleic acid.

In some instances, the methods involve hybridization and/or amplification assays that include, but are not limited to, Southern or Northern analyses, polymerase chain reaction analyses, and probe arrays. Non-limiting amplification reactions include, but are not limited to, qPCR, self-sustained sequence replication, transcriptional amplification system, Q-Beta Replicase, rolling circle replication, or any other nucleic acid amplification known in the art. As discussed, reference to qPCR herein includes use of TaqMan™ methods. An additional exemplary hybridization assay includes the use of nucleic acid probes conjugated or otherwise immobilized on a bead, multi-well plate, or other substrate, wherein the nucleic acid probes are configured to hybridize with a target nucleic acid sequence of a genotype provided herein. A non-limiting method is one employed in Anal Chem. 2013 Feb. 5; 85(3):1932-9.

In some embodiments, detecting the presence or absence of a genotype comprises sequencing genetic material from the subject. Sequencing can be performed with any appropriate sequencing technology, including but not limited to single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis. Sequencing methods also include next-generation sequencing, e.g., modern sequencing technologies such as Illumina sequencing (e.g., Solexa), Roche 454 sequencing, Ion torrent sequencing, and SOLID sequencing. In some cases, next-generation sequencing involves high-throughput sequencing methods. Additional sequencing methods available to one of skill in the art may also be employed.

In some instances, a number of nucleotides that are sequenced are at least 5, 10, 15, 20, 25, 30, 35, 40, 45, 50, 100, 150, 200, 300, 400, 500, 2000, 4000, 6000, 8000, 10000, 20000, 50000, 100000, or more than 100000 nucleotides. In some instances, the number of nucleotides sequenced is in a range of about 1 to about 100000 nucleotides, about 1 to about 10000 nucleotides, about 1 to about 1000 nucleotides, about 1 to about 500 nucleotides, about 1 to about 300 nucleotides, about 1 to about 200 nucleotides, about 1 to about 100 nucleotides, about 5 to about 100000 nucleotides, about 5 to about 10000 nucleotides, about 5 to about 1000 nucleotides, about 5 to about 500 nucleotides, about 5 to about 300 nucleotides, about 5 to about 200 nucleotides, about 5 to about 100 nucleotides, about 10 to about 100000 nucleotides, about 10 to about 10000 nucleotides, about 10 to about 1000 nucleotides, about 10 to about 500 nucleotides, about 10 to about 300 nucleotides, about 10 to about 200 nucleotides, about 10 to about 100 nucleotides, about 20 to about 100000 nucleotides, about 20 to about 10000 nucleotides, about 20 to about 1000 nucleotides, about 20 to about 500 nucleotides, about 20 to about 300 nucleotides, about 20 to about 200 nucleotides, about 20 to about 100 nucleotides, about 30 to about 100000 nucleotides, about 30 to about 10000 nucleotides, about 30 to about 1000 nucleotides, about 30 to about 500 nucleotides, about 30 to about 300 nucleotides, about 30 to about 200 nucleotides, about 30 to about 100 nucleotides, about 50 to about 100000 nucleotides, about 50 to about 10000 nucleotides, about 50 to about 1000 nucleotides, about 50 to about 500 nucleotides, about 50 to about 300 nucleotides, about 50 to about 200 nucleotides, or about 50 to about 100 nucleotides.

Exemplary probes comprise a nucleic acid sequence of at least 10 contiguous nucleic acids for any one of rs3810936, rs6478 108, rs6478109, rs7848647, or rs7869487, or a reverse complement thereof. In some embodiments, the nucleic acid sequence comprises a variant nucleotide of interest and at least a portion of one or more flanking sequences (flanking the variant nucleotide of interest). In some embodiments, the variant nucleotide of interest at rs3810936, rs6478108, rs6478109, rs7848647, or rs7869487 is indicated with a non-nucleobase letter (e.g., R, N, S) in National Library of Medicine (NCBI) dsSNP database, which flanking sequences are hereby incorporated by reference in their entirety. In some embodiments, the probe comprises at least 70%, 80%, 85%, 90%, 91%, 92%, 93%, 94%, 95%, 96%, 97%, 98% or 99% sequence identity to a sequence comprising any one of rs3810936, rs6478108, rs6478 109, rs7848647, or rs7869487, or its reverse complement. In some instances, forward and reverse primers are used to amplify the target nucleic acid sequence. Forward and reverse primers may comprise a nucleic acid sequence flanking the variant nucleotide of interest corresponding to the nucleic acid sequence provided in any one of rs3810936, rs6478 108, rs6478109, rs7848647, or rs7869487, or a reverse complement thereof. In some embodiments, the variant nucleotide of interest comprises a “C” at rs3810936, a “T” at rs6478108, a “G” at rs6478109, a “C” at rs7848647, or a “T” at rs7869487, or any combination thereof. In some embodiments, the variant nucleotide of interest comprises a “T” at rs3810936, a “C” at rs6478 108, a “A” at rs6478 109, a “T” at rs7848647, a “C” at rs7869487, or any combination thereof. In some embodiments, the variant nucleotide of interest comprises a “T” at rs3810936, a “C” at rs6478108, a “A” at rs6478109, a “T” at rs7848647, or a “T” at rs7869487, or any combination thereof.

Examples of molecules that are utilized as probes include, but are not limited to, RNA and DNA. In some embodiments, the term “probe” with regards to nucleic acids, refers to any molecule that is capable of selectively binding to a specifically intended target nucleic acid sequence. In some instances, probes are specifically designed to be labeled, for example, with a radioactive label, a fluorescent label, an enzyme, a chemiluminescent tag, a colorimetric tag, or other labels or tags that are known in the art. In some instances, the fluorescent label comprises a fluorophore. In some instances, the fluorophore is an aromatic or heteroaromatic compound. In some instances, the fluorophore is a pyrene, anthracene, naphthalene, acridine, stilbene, benzoxazole, indole, benzindole, oxazole, thiazole, benzothiazole, canine, carbocyanine, salicylate, anthranilate, xanthenes dye, coumarin. Exemplary xanthene dyes include, e.g., fluorescein and rhodamine dyes. Fluorescein and rhodamine dyes include, but are not limited to 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Suitable fluorescent probes also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Exemplary coumarins include, e.g., 3-phenyl-7-isocyanatocoumarin; acridines, such as 9-isothiocyanatoacridine and acridine orange; N-(p-(2-benzoxazolyl)phenyl) maleimide; cyanines, such as, e.g., indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno [2,3, 4-ij: 5,6, 7-i′j′] diquinolizin-18-ium, 9-[2 (or 4)-[[[6-[2,5-dioxo-1-pyrrolidinyl)oxy]-6-oxohexyl]amino] sulfonyl]-4 (or 2)-sulfophenyl]-2,3, 6,7, 12,13, 16,17-octahydro-inner salt (TR or Texas Red); or BODIPY™ dyes. In some cases, the probe comprises FAM as the dye label.

In some instances, primers and/or probes described herein for detecting a target nucleic acid are used in an amplification reaction. In some instances, the amplification reaction is qPCR. An exemplary qPCR is a method employing a TaqMan™ assay. “Wt_Probe Hex” and “Mut Probe_FAM” mean “Wild type_probes_tagged with HEX reporter dye” and “Mut_probe_tagged with FAM reporter dye”, respectively. “+” stands for LNA bases (Locked nucleotides), which are analogues that are modified at 2′-0, 4′-C and form a bridge. This bridge results in restricted base pairing giving room to adjust the Tm as needed between the probes. Thus, +A, +T, +C or +G signify A, T, G or C bases are added on the modified backbone.

In some instances, qPCR comprises using an intercalating dye. Examples of intercalating dyes include SYBR green I, SYBR green II, SYBR gold, ethidium bromide, methylene blue, Pyronin Y, DAPI, acridine orange, Blue View or phycoerythrin. In some instances, the intercalating dye is SYBR.

In some instances, a number of amplification cycles for detecting a target nucleic acid in an amplification assay is about 5 to about 30 cycles. In some instances, the number of amplification cycles for detecting a target nucleic acid is at least about 5 cycles. In some instances, the number of amplification cycles for detecting a target nucleic acid is at most about 30 cycles. In some instances, the number of amplification cycles for detecting a target nucleic acid is about 5 to about 10, about 5 to about 15, about 5 to about 20, about 5 to about 25, about 5 to about 30, about 10 to about 15, about 10 to about 20, about 10 to about 25, about 10 to about 30, about 15 to about 20, about 15 to about 25, about 15 to about 30, about 20 to about 25, about 20 to about 30, or about 25 to about 30 cycles.

In one aspect, the methods provided herein for determining the presence, absence, and/or quantity of a nucleic acid sequence from a particular genotype comprise an amplification reaction such as qPCR. In an exemplary method, genetic material is obtained from a sample of a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol, or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification.

In the exemplary qPCR assay, the nucleic acid sample is combined with primers and probes specific for a target nucleic acid that may or may not be present in the sample, and a DNA polymerase. An amplification reaction is performed with a thermal cycler that heats and cools the sample for nucleic acid amplification, and illuminates the sample at a specific wavelength to excite a fluorophore on the probe and detect the emitted fluorescence. For TaqMan™ methods, the probe may be a hydroly sable probe comprising a fluorophore and quencher that is hydrolyzed by DNA polymerase when hybridized to a target nucleic acid. In some cases, the presence of a target nucleic acid is determined when the number of amplification cycles to reach a threshold value is less than 30, 29, 28, 27, 26, 25, 24, 23, 22, 21, or 20 cycles.

In some embodiments, one target nucleic acid (e.g., a polymorphism) is detected with the methods disclosed herein. In some embodiments, at least 2, 3, 4, 5, 6, 7, 8, 9, or 10 target nucleic acids are detected. In some embodiments, the at least 2, 3, 4, 5, 6, 7, 8, 9, or 10 target nucleic acids are detected in a single multiplexed assay.

In some embodiments, the one or more SNPs belong to a haplotype at a locus in TNFSF15. In some embodiments, the one or more SNPs belong to a haplotype described in Michelsen K S, Thomas L S, Taylor K D, Yu Q T, Mei L, Landers C J, Derkowski C, McGovern D P, Rotter J I, Targan S R. IBD-associated TL1A gene (TNFSF15) haplotypes determine increased expression of TL1A protein. PLOS One. 2009; 4 (3): e4719, which is incorporated herein by reference. In some embodiments, the one or more SNPs comprises rs3810936, rs6478 108, rs6478109, rs7848647, or rs7869487, or any combination thereof. In some embodiments, the one or more SNPs comprises a “C” at rs3810936, a “T” at rs6478108, a “G” at rs6478109, a “C” at rs7848647, or a “T” atrs7869487, or any combination thereof. In some embodiments, the one or more SNPs comprises a “T” at rs3810936, a “C” at rs6478108, a “A” at rs6478 109, a “T” at rs7848647, a “C” atrs7869487, or any combination thereof. In some embodiments, the one or more SNPs comprises a “T” at rs3810936, a “C” at rs6478108, a “A” at rs6478 109, a “T” at rs7848647, or a “T” at rs7869487, or any combination thereof.

Disclosed herein, in some embodiments, are methods, systems and kits for measuring an increase in expression of a gene expression product of INFSF15 or TNFSF25 in the one or more samples obtained from the subject. In one embodiment, the increase in the expression of the gene expression product of TNFSF15 is measured in a sample comprising peripheral blood mononuclear cells (PBMCs). In some embodiments, the PBMCs are obtained from the sample with immune complex under conditions sufficient for the PBMCs to express the gene expression product. In some embodiments, the method comprises measuring the gene expression product. In some embodiments, the method comprises comparing the expression of the gene expression product with the reference expression level. In some embodiments, the expression of the gene expression product is measured by a process comprising single molecule protein detection. The reference expression level may be an expression level obtained from a subject without an IBD. The reference expression level may be an expression level obtained from a subject without the ileitis, intestinal fibrosis or the colitis.

In some embodiments, the gene expression product is a protein. In some embodiments, the protein is a soluble protein. In some embodiments, the gene expression product is RNA. In some embodiments, the gene expression product is mRNA encoding TL1A. In some embodiments, the gene expression product is TL1A. In some embodiments, the gene expression product is mRNA encoding DR3 In some embodiments, the gene expression product is DR3.

In some embodiments, the subject is predicted to develop the ileitis, the intestinal fibrosis, or the colitis based, at least in part, on an increase in expression of a gene expression product of TNFSF15 or TNFRSF25 in the one or more samples obtained from the subject relative to the reference expression level obtained from the reference subject. In some embodiments, the methods further comprise measuring the increase in the expression of a gene expression product of TNFRSF15 or TNFRSF25 in the one or more samples relative to a reference expression level obtained from a reference subject without the IBD. In some embodiments, gene expression products of both TNFRSF15 and TNFRSF25 are measured. In some embodiments, the subject is predicted to develop the ileitis, the intestinal fibrosis, or the colitis based, at least in part, on an increase in expression of a gene expression product of TNFSF15 and TNFRSF25 in the one or more samples obtained from the subject relative to the reference expression level obtained from the reference subject

To practice the methods and systems provided herein, genetic material may be extracted from a sample obtained from a subject, e.g., a sample of blood or serum. In certain embodiments where nucleic acids are extracted, the nucleic acids are extracted using any technique that does not interfere with subsequent analysis. In certain embodiments, this technique uses alcohol precipitation using ethanol, methanol or isopropyl alcohol. In certain embodiments, this technique uses phenol, chloroform, or any combination thereof. In certain embodiments, this technique uses cesium chloride. In certain embodiments, this technique uses sodium, potassium or ammonium acetate or any other salt commonly used to precipitate DNA. In certain embodiments, this technique utilizes a column or resin based nucleic acid purification scheme such as those commonly sold commercially, one non-limiting example would be the GenElute Bacterial Genomic DNA Kit available from Sigma Aldrich. In certain embodiments, after extraction the nucleic acid is stored in water, Tris buffer, or Tris-EDTA buffer before subsequent analysis. In an exemplary embodiment, the nucleic acid material is extracted in water. In some cases, extraction does not comprise nucleic acid purification. In certain embodiments, RNA may be extracted from cells using RNA extraction techniques including, for example, using acid phenol/guanidine isothiocyanate extraction (RNAzol B; Biogenesis), RNeasy RNA preparation kits (Qiagen) or PAXgene (PreAnalytix, Switzerland).

In some embodiments, methods of detecting a presence, absence, or level of a target protein (e.g., biomarker) in the sample obtained from the subject involve detecting protein activity or expression. In some embodiments, the target protein is TL1A, or a binding partner of TL1A such as Death Domain Receptor 3 (DcR3) or DR3. A target protein may be detected by use of an antibody-based assay, where an antibody specific to the target protein is utilized. In some embodiments, antibody-based detection methods utilize an antibody that binds to any region of target protein. An exemplary method of analysis comprises performing an enzyme-linked immunosorbent assay (ELISA). The ELISA assay may be a sandwich ELISA or a direct ELISA. Another exemplary method of analysis comprises a single molecule array, e.g., Simoa. Other exemplary methods of detection include immunohistochemistry and lateral flow assay. Additional exemplary methods for detecting target protein include, but are not limited to, gel electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (RIA), immunofluorescent assays, and Western blotting. In some embodiments, antibodies, or antibody fragments, are used in methods such as Western blots or immunofluorescence techniques to detect the expressed proteins. The antibody or protein can be immobilized on a solid support for Western blots and immunofluorescence techniques. Suitable solid phase supports or carriers include any support capable of binding an antigen or an antibody. Exemplary supports or carriers include glass, polystyrene, polypropylene, polyethylene, dextran, nylon, amylases, natural and modified celluloses, polyacrylamides, gabbros, and magnetite.

In some cases, a target protein may be detected by detecting binding between the target protein and a binding partner of the target protein. Non-limiting examples of binding partners to TL1A include DcR3, and DR3. Exemplary methods of analysis of protein-protein binding comprise performing an assay in vivo or in vitro, or ex vivo. In some instances, the method of analysis comprises an assay such as a co-immunoprecipitation (co-IP), pull-down, crosslinking protein interaction analysis, labeled transfer protein interaction analysis, or Far-western blot analysis, FRET based assay, including, for example FRET-FLIM, a yeast two-hybrid assay, BiFC, or split luciferase assay.

Any suitable method for detecting a target protein or biomarker disclosed herein may be used to detect a presence, absence, or level of a serological marker. In some embodiments, the presence or the level of the one or more serological markers is detected using an enzyme-linked immunosorbent assay (ELISA), a single molecule array (Simoa), immunohistochemistry, internal transcribed spacer (ITS) sequencing, or any combination thereof. In some embodiments, the ELISA is a fixed leukocyte ELISA. In some embodiments, the ELISA is a fixed neutrophil ELISA. A fixed leukocyte or neutrophil ELISA may be useful for the detection of certain serological markers, such as those described in Saxon et al., A distinct subset of antineutrophil cytoplasmic antibodies is associated with inflammatory bowel disease, J. Allergy Clin. Immuno. 86:2; 202-210 (August 1990). In some embodiments, ELISA units (EU) are used to measure positivity of a presence or level of a serological marker (e.g., seropositivity), which reflects a percentage of a standard or reference value. In some embodiments, the standard comprises pooled sera obtained from well-characterized patient population (e.g., diagnosed with the same disease or condition the subject has, or is suspected of having) reported as being seropositive for the serological marker of interest. In some embodiments, the control or reference value comprises 10, 20, 30, 40, 50, 60, 70, 80, 90, or 100 EU. In some instances, a quartile sum scores are calculated using, for example, the methods reported in Landers C J, Cohavy O, Misra R. et al., Selected loss of tolerance evidenced by Crohn's disease-associated immune responses to auto- and microbial antigens. Gastroenterology (2002)123:689-699.

### Treating a Disease or a Condition

Disclosed herein are systems, methods and kits for treating a disease or condition, or a symptom of the disease or condition, in a subject, comprising administrating of therapeutic effective amount of one or more therapeutic agents to the subject. In some embodiments, the subject is selected for treatment with the one or more therapeutic agents based, at least in part, on a presence of one or more genetic risk variants, an increased level of one or more biomarkers or serological markers, or any combination thereof, with reference to a reference subject that does have the disease or the condition. In some embodiments, the disease or condition comprises an immune-mediated inflammatory disease, such as inflammatory bowel disease. In some embodiments, the IBD comprises Crohn's disease or ulcerative colitis. In some embodiments, the disease or the condition comprises intestinal fibrosis. In some embodiments, the disease or the condition comprises colitis. In some embodiments, the disease or the condition comprises ileitis. In some embodiments, the subject has two or more of the diseases or the conditions disclosed herein. In some embodiments, the one or more therapeutic agents is administered to the subject alone (e.g., standalone therapy). In some embodiments, the one or more therapeutic agents is administered in combination with an additional agent. In some embodiments, the therapeutic agent is a first-line therapy for the disease or condition. In some embodiments, the therapeutic agent is a second-line, third-line, or fourth-line therapy, for the disease or condition.

In certain aspects, described herein is a method of treating inflammatory bowel disease (IBD) in a subject, the method comprising: administering to the subject a therapeutic agent as described herein, wherein the subject is predicted to develop ileitis, intestinal fibrosis, or colitis based, at least in part, on an increase in an amount of the acetate or the metabolite thereof, or an amount of the acetate producing bacteria, relative to a reference level in a reference subject that does not have the IBD detected in one or more samples obtained from the subject. In some embodiments, the therapeutic agent comprises an active agent to reduce an amount of acetate or a metabolite thereof, or of an acetate producing bacteria. In some embodiments, the therapeutic agent comprises an inhibitor of TL1A activity or expression. In some embodiments, the inhibitor of TL1A activity or expression comprises an anti-TL1A antibody or antigen-binding fragment thereof.

In certain aspects, described herein is a method of treating ileitis, intestinal fibrosis, or colitis in a subject, the method comprising: administering to the subject a therapeutic agent as described herein, wherein the subject is predicted to develop the ileitis, the intestinal fibrosis, or the colitis based, at least in part, on an increase in an amount of the acetate or the metabolite thereof, or an amount of the acetate producing bacteria, relative to a reference level in a reference subject that does not have the ileitis, the intestinal fibrosis, or the colitis detected in one or more samples obtained from the subject. In some embodiments, the therapeutic agent comprises an active agent to reduce an amount of acetate or a metabolite thereof, or of an acetate producing bacteria. In some embodiments, the therapeutic agent comprises an inhibitor of TL1A activity or expression. In some embodiments, the inhibitor of TL1A activity or expression comprises an anti-TL1A antibody or antigen-binding fragment thereof.

In some embodiments, the methods further comprise measuring an increase in expression of a gene expression product of TNFSF15 in the one or more samples obtained from the subject. In some embodiments, the gene expression product of TNFSF15 is TL1A. In some embodiments, the gene expression product of TNFSF15 is mRNA encoding TL1A. In some embodiments, the methods further comprise measuring an increase in expression of a gene expression product of TNFSF25 in the one or more samples obtained from the subject. In some embodiments, the gene expression product of TNFSF25 is DR3. In some embodiments, the gene expression product of TNFSF25 is mRNA encoding DR3. In some embodiments, the methods further comprise detecting a presence of one or more genetic risk variants in the one or more samples obtained from the subject, such as for example, one or more genetic risk variants at TNFSF15 disclosed herein. In some embodiments, the one or more genetic risk variants comprises one or more single nucleotide polymorphisms (SNPs). In some embodiments, the one or more SNPs belongs to a haplotype at TNFSF15. In some embodiments, the one more SNPs comprises rs3810936, rs6478108, rs6478109, rs7848647, or rs7869487, or a SNP in linkage disequilibrium (LD) thereof, or any combination thereof. In some embodiments, the one more SNPs comprises rs3810936. In some embodiments, the one more SNPs comprises rs6478108. In some embodiments, the one more SNPs comprises rs6478109. In some embodiments, the one more SNPs comprises rs7848647. In some embodiments, the one more SNPs comprises rs7869487. In some embodiments, the one or more SNPs comprises a “C” at rs3810936, a “T” at rs6478108, a “G” at rs6478109, a “C” at rs7848647, or a “T” at rs7869487, or any combination thereof. In some embodiments, the one or more SNPs comprises a “T” at rs3810936, a “C” at rs6478108, a “A” at rs6478109, a “T” at rs7848647, a “C” at rs7869487, or any combination thereof. In some embodiments, the one or more SNPs comprises a “T” at rs3810936, a “C” at rs6478108, a “A” at rs6478 109, a “T” at rs7848647, or a “T” at rs7869487, or any combination thereof. In some embodiments, LD is characterized with a r2 of at least about 0.80. In some embodiments, the methods further comprise measuring an increase in production of interferon gamma (IFN-gamma) in the one or more samples obtained from the subject. In some embodiments, the methods further comprise measuring a decrease in production of protective short-chain fatty acids (e.g., butyrate, propionate) in the one or more samples obtained from the subject. In some embodiments, the methods further comprise detecting an abnormal morphology of a cell in the one or more samples obtained from the subject, such as a Paneth cell. In some embodiments, the abnormal morphology comprises a distended or distorted endoplasmic reticulum, a distended or distorted mitochondria, or an increased vesiculation, or a combination thereof. In some embodiments, the method further comprises detecting hyperplasia in the cell or a population of the cells (e.g., Paneth cells) obtained from the one or more samples obtained from the subject. In some embodiments, the method further comprises measure an increase in a production of bile acids in the one or more samples obtained from the subject. In some embodiments, the method comprises diagnosing the subject with ileitis, intestinal fibrosis, or colitis based, at least in part, one or more of the above biomarkers (e.g., TL1A, DR3, IFN-gamma, one or more TNFSF15 genetic risk variants, protective short-chain fatty acid, Paneth cell morphology or hyperplasia or both, bile acids, and so on). In some embodiments, the method comprises predicting that the subject will develop the ileitis, intestinal fibrosis, or colitis based, at least in part, one or more of the above biomarkers. In some embodiments, the increase or the decrease of the above biomarkers is relative to a reference sample obtained from a reference subject or plurality of reference subjects. In some embodiments, the reference subject does not have the ileitis, intestinal fibrosis, or colitis. In some embodiments, the reference subject does not have an inflammatory bowel disease. In some embodiments, the reference subject is healthy. In some embodiments, the methods further comprise administering to the subject one or more therapeutic agents to treat or prevent the ileitis, intestinal fibrosis, or colitis.

### Therapeutic Agents

In one aspect, provided herein are therapeutic agents for treatment a disease or a condition disclosed herein. In some embodiments, the disease or the condition comprises inflammatory bowel disease (IBD). In some embodiments, the IBD comprises Crohn's disease (CD) or ulcerative colitis. In some embodiments, the disease or the condition comprises ileitis, intestinal fibrosis, or colitis. In some embodiments, the therapeutic agents is or comprises a small molecule drug, an antibody or antigen-binding fragment thereof, a protein therapy, or a cellular therapy, or a combination thereof.

In some embodiments, the therapeutic agent comprises an antibody and antigen-binding fragment. In some embodiments, an antibody comprises an antigen-binding fragment that refers to a portion of an antibody having antigenic determining variable regions of an antibody. Examples of antigen-binding fragments include, but are not limited to, Fab, Fab′, F(ab′)₂, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′) 2, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region. The humanized heavy chain variable domain may comprise IGHV1-46*02 framework with no or fewer than about 10, 9, 8, 7, 6, 5, 4, 3, 2, or 1 amino acid mutations. The humanized light chain variable domain may comprise IGKV3-20 framework with no or fewer than about 10, 9, 8, 7, 6, 5, 4, 3, 2, or 1 amino acid mutations.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

In some embodiments, an antibody that specifically binds to a protein indicates that the antibody reacts or associates more frequently, more rapidly, with greater duration, with greater affinity, or with some combination of the above to the protein than with alternative substances, including unrelated proteins.

In some embodiments, the terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as fusion with another polypeptide and/or conjugation, e.g., with a labeling component. Also included within the definition are, for example, polypeptides containing one or more analogs of an amino acid (for example, unnatural amino acids, etc.), as well as other modifications known in the art.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In some embodiments, the term “about” means within 10% of the stated amount. For instance, an antibody variable region comprising about 80% identity to a reference variable region may comprise 72% to 88% identity to the reference variable region.

In some embodiments, the therapeutic agent comprises an anti-tumor necrosis factor (TNF) therapy, an anti-TL1A therapy, anti-integrin therapy, small molecules (e.g., tofacitinib), selective janus kinase (JAK) inhibitors, anti-interleukin (IL) (e.g., IL-12/IL-23), leukocyte trafficking/migrating inhibitors (e.g., as sphingosine-1-phosphate receptor modulator. In some embodiments, the therapeutic agents provided in Badr Al-Bawardy et al., Novel and Emerging Therapies for Inflammatory Bowel Disease. Front. Pharmacol. 12:651415, which is hereby incorporated by reference in its entirety. In some embodiments, the therapeutic agent is an inhibitor of a acetate or an acetate producing bacteria.

### Acetate and Acetate-Producing Bacteria Inhibitors

In certain embodiments, described herein is an active agent that is effective to reduce the amount of the acetate-producing bacteria in the subject. The active agent may comprise a small molecule drug, an antibody or antigen binding fragment thereof, an inhibitor, or an enzyme or a catalytically active portion thereof.

The active agent may comprise an antibiotic, a probiotic, a prebiotic, or any combination thereof. In some embodiments, the active agent comprises an antibiotic. In some embodiments, the antibiotic comprises a cephalosporin, a fluoroquinolone, a carbapenem, a colistin, an aminoglycoside, vancomycin, streptomycin, or methicillin. In some embodiments, the active agent comprises a prebiotic. In some embodiments, the active agent comprises a probiotic.

In some embodiments, the probiotic is administered concurrently with the antibiotic. In some embodiments, the probiotic is administered following the antibiotic. In some embodiments, the probiotic is administered concurrently with the prebiotic.

In some embodiments, the active agent comprises a fecal microbiota transplant. “Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the fecal microbiota transplant is administered with colonic administration. In some embodiments, the fecal microbiota transplant comprises a supplement derived from a stool sample from an individual that does not have the IBD.

In some embodiments, the fecal microbiota transplant comprises bacteria that produce acetate in an amount comprising about 50 mmol/l. In some embodiments, the fecal microbiota transplant comprises bacteria that produce acetate in an amount comprising at least about 10 mmol/l, about 20 mmol/l, about 30 mmol/l, about 40 mmol/l, about 50 mmol/l, about 60 mmol/l, about 70 mmol/l, about 80 mmol/l, about 90 mmol/l, or about 100 mmol/l. In some embodiments, the fecal microbiota transplant comprises bacteria that produce acetate in an amount comprising no more than about 10 mmol/l, about 20 mmol/l, about 30 mmol/l, about 40 mmol/l, about 50 mmol/l, about 60 mmol/l, about 70 mmol/l, about 80 mmol/l, about 90 mmol/l, about 100 mmol/l, about 110 mmol/l, about 120 mmol/l, about 130 mmol/l, about 140 mmol/l, about 150 mmol/l, about 160 mmol/l, about 170 mmol/l, about 180 mmol/l, about 190 mmol/l, or about 200 mmol/l. In some embodiment, the fecal microbiota transplant comprises bacteria that produce acetate in an amount comprising from about 10 mmol/l to about 50 mmol/l, about 20 mmol/l to about 70 mmol/l, about 30 mmol/l to about 80 mmol/l, about 40 mmol/l to about 90 mmol/l, or about 50 mmol/l to about 100 mmol/l.

In some embodiments, the active agent comprises a chelating agent. In some embodiments, the chelating agent is administering with colonic administration. In some embodiments, the chelating agent is configured to bind acetate. In some embodiments, the chelating agent is configured to bind a metabolite of acetate. In some embodiments, the metabolite of acetate comprises 3-hydroxy-propionate.

In some embodiments, the method comprises prescribing a change in a diet of the subject. In some embodiments, the change in the diet comprises reducing the subject's intake of acidic food, vinegar, acetic acid, carbohydrates, sugars, starches, probiotic-containing foods, or any combination thereof. In some embodiments, the active agent is a butyrate supplement.

In some embodiments, wherein the active agent comprises an inhibitor. In some embodiments, the small molecule drug is therapeutically effective to disrupt the production of the acetate or the metabolite thereof in the subject by the acetate producing bacteria. In some embodiments, the antibody or antigen binding fragment thereof is therapeutically effective to bind to the acetate or the metabolite thereof.

In some embodiments, the enzyme or the catalytically active portion thereof promotes the metabolism of acetate or the metabolite thereof in the subject. In some embodiments, the enzyme comprises coenzyme A, acetyl-coenzyme A, formate acetyl transferase, or any combination thereof.

In some embodiments, the active agent is an inhibitor of G-protein-coupled receptor 43 (GPR43).

### TL1A Therapies

In some embodiments, the therapeutic agents comprise a Tumor necrosis factor-like cytokine 1A (TL1A) therapy. In some embodiments, the TL1A therapy is a modulator of TL1A activity or expression. In some embodiments, the TL1A therapy is an inhibitor of TL1A activity or expression. In some embodiments, the TL1A therapy is an allosteric modulator of TL1A. Non-limiting examples of an inhibitor of TL1A expression include RNA to protein translation inhibitors, antisense oligonucleotides targeting the TNFSF15 mRNA (such as miRNAs, or siRNA), epigenetic editing (such as, for example, targeting the DNA-binding domain of TNFSF15, or post-translational modifications of histone tails and/or DNA molecules). Non-limiting examples of an inhibitor of TL1A activity include antagonists to the TL1A binding partners (e.g., Decoy Receptor 3 (DcR3)), antagonists to TL1A antigen, and antagonists to gene expression products involved in TL1A mediated disease. Antagonists as disclosed herein, may include, but are not limited to, an anti-TL1A antibody or antigen-binding fragment thereof, or a small molecule drug. In some embodiments, the TL1A therapy comprises an anti-TL1A therapy. In one embodiment, the anti-TL1A therapy is a small molecule drug. In one embodiment, the anti-TL1A therapy is a biologic drug. In some embodiments, the anti-TL1A therapy comprises an antibody and antigen-binding fragment thereof. In some embodiments, an antibody comprises an antigen-binding fragment that refers to a portion of an antibody having antigenic determining variable regions of an antibody. Examples of antigen-binding fragments include, but are not limited to, Fab, Fab′, F(ab′)2, and Fv fragments, linear antibodies, single chain antibodies, and multispecific antibodies formed from antibody fragments. In some embodiments, an antibody refers to an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. In some embodiments, an antibody includes intact polyclonal antibodies, intact monoclonal antibodies, antibody fragments (such as Fab, Fab′, F(ab′)2, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, or the like.

In some embodiments, the anti-TL1A antibody or antigen-binding fragment thereof is humanized. In some embodiments, a humanized antibody refers to forms of non-human (e.g., murine) antibodies having specific immunoglobulin chains, chimeric immunoglobulins, or fragments thereof that contain minimal non-human (e.g., murine) sequences. In a non-limiting example, a humanized antibody comprises less than about 40% non-human sequence in the variable region. In some cases, a humanized antibody comprises less than about 20% non-human sequence in a full-length antibody sequence. In a further non-limiting example, a humanized antibody comprises less than about 20% non-human sequence in the framework region of each of the heavy chain and light chain variable regions. For instance, the humanized antibody comprises less than about 20%, 19%, 18%, 17%, 16%, 15%, 14%, 13%, 12%, 11%, 10%, 9%, 8%, 7%, 6%, 5%, 4%, 3%, 2%, or 1% non-human sequence in the framework region of each of the heavy chain and light chain variable regions. As another example, the humanized antibody comprises about or less than about 15, 14, 13, 12, 11, 10, 9, 8, 7, 6, 5, 4, 3, 2, or 1 non-human sequences in the framework region of each of the heavy chain and light chain variable regions. In some cases, humanized antibodies are human immunoglobulins in which residues from the complementarity determining region (CDR) are replaced by residues from the CDR of a non-human species (e.g., mouse, rat, rabbit, hamster) that have the desired specificity, affinity, and capability. These humanized antibodies may contain one or more non-human species mutations, e.g., the heavy chain comprises about 1, 2, 3, 4, 5, 6, 7, 8, 9, 10, 11, 12, 13, 14, or 15 non-human species mutations in the framework region, and the light chain comprises about 1, 2, 3, 4, 5, 6, 7, 8, 9, 10, 11, 12, 13, 14, or 15 non-human species mutations in the framework region.

In some embodiments, the anti-TL1A antibody or antigen-binding fragment thereof is a chimeric antibody or antigen-binding fragment thereof. In some embodiments, chimeric antibodies refer to antibodies wherein the sequence of the immunoglobulin molecule is derived from two or more species. As a non-limiting example, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

In some embodiments, the anti-TL1A antibody or antigen-binding fragment thereof specifically binds to TL1A (e.g., Entrez Gene: 9966; UniProtKB: 095150). In some embodiments, the anti-TL1A antibody or antigen-binding fragment thereof specifically binds to soluble TL1A. In some embodiments, the anti-TL1A antibody or antigen-binding fragment thereof specifically binds to membrane bound TL1A. In some embodiments, an anti-TL1A antibody or antigen-binding fragment thereof comprises a heavy chain comprising four heavy chain framework regions (HCFR) and three heavy chain complementarity-determining regions (HCDR): HCFR1, HCDR1, HCFR2, HCDR2, HCFR3, HCDR3, and HCFR4; and a light chain comprising four light chain framework regions (LCFR) and three light chain complementarity-determining regions (LCDR): LCFR1, LCDR1, LCFR2, LCDR2, LCFR3, LCDR3, and LCFR4. In some embodiments, the anti-TL1A antibody or antigen-binding fragment thereof is or comprises PRA023 (such as, for example, disclosed in clinical trial NCT05013905, NCT05270668, or NCT04996797). In some embodiments, the anti-TL1A antibody or antigen-binding fragment thereof is or comprises PF-06480605 (such as, for example, disclosed in clinical trial NCT04090411, NCT05471492, or NCT02840721). In some embodiments, the anti-TL1A antibody or antigen-binding fragment thereof is or comprises TEV-48574 (such as, for example, disclosed in clinical trial NCT05499130). In some embodiments, the anti-TL1A antibody or antigen-binding fragment thereof is or comprises an antibody or antigen-binding fragment disclosed in U.S. Pat. Nos. 10,322,174; 10,689,439; 11,440,954; 11,136,386; 11,292,848; 9,683,998; 10,968,279; 8,642,741; 10,822,422; 8,263,743; 8,728,482; 10,138,296; 9,290,576; or 11,104,745; or any combination thereof; each of which is hereby incorporated by reference in its entirety.

### Pharmaceutical Composition

A pharmaceutical composition, as used herein, refers to a mixture of a therapeutic agent, with other chemical components (i.e. pharmaceutically acceptable inactive ingredients), such as carriers, excipients, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, or one or more combination thereof. Optionally, the compositions include two or more therapeutic agent (e.g., one or more therapeutic agents and one or more additional agents) as discussed herein. In practicing the methods of treatment or use provided herein, therapeutically effective amounts of therapeutic agents described herein are administered in a pharmaceutical composition to a mammal having a disease, disorder, or condition to be treated, e.g., an inflammatory disease, fibrostenotic disease, and/or fibrotic disease. In some embodiments, the mammal is a human. A therapeutically effective amount can vary widely depending on the severity of the disease, the age and relative health of the subject, the potency of the therapeutic agent used and other factors. The therapeutic agents can be used singly or in combination with one or more therapeutic agents as components of mixtures.

The pharmaceutical formulations described herein are administered to a subject by appropriate administration routes, including but not limited to, intravenous, intraarterial, oral, parenteral, buccal, topical, transdermal, rectal, intramuscular, subcutaneous, intraosseous, transmucosal, inhalation, or intraperitoneal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

Pharmaceutical compositions including a therapeutic agent are manufactured in a conventional manner, such as, by way of example only, by means of conventional mixing, dissolving, granulating, dragee-making, levigating, emulsifying, encapsulating, entrapping or compression processes.

The pharmaceutical compositions may include at least a therapeutic agent as an active ingredient in free-acid or free-base form, or in a pharmaceutically acceptable salt form. In addition, the methods and pharmaceutical compositions described herein include the use of N-oxides (if appropriate), crystalline forms, amorphous phases, as well as active metabolites of these compounds having the same type of activity. In some embodiments, therapeutic agents exist in unsolvated form or in solvated forms with pharmaceutically acceptable solvents such as water, ethanol, and the like. The solvated forms of the therapeutic agents are also considered to be disclosed herein.

In some embodiments, a therapeutic agent exists as a tautomer. All tautomers are included within the scope of the agents presented herein. As such, it is to be understood that a therapeutic agent or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound.

In some embodiments, a therapeutic agent exists as an enantiomer, diastereomer, or other stereoisomeric form. The agents disclosed herein include all enantiomeric, diastereomeric, and epimeric forms as well as mixtures thereof.

In some embodiments, therapeutic agents described herein may be prepared as prodrugs. A “prodrug” refers to an agent that is converted into the parent drug in vivo. Prodrugs are often useful because, in some situations, they may be easier to administer than the parent drug. They may, for instance, be bioavailable by oral administration whereas the parent is not. The prodrug may also have improved solubility in pharmaceutical compositions over the parent drug. An example, without limitation, of a prodrug would be a therapeutic agent described herein, which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. A further example of a prodrug might be a short peptide (polyaminoacid) bonded to an acid group where the peptide is metabolized to reveal the active moiety. In certain embodiments, upon in vivo administration, a prodrug is chemically converted to the biologically, pharmaceutically or therapeutically active form of the therapeutic agent. In certain embodiments, a prodrug is enzymatically metabolized by one or more steps or processes to the biologically, pharmaceutically or therapeutically active form of the therapeutic agent.

Prodrug forms of the therapeutic agents, wherein the prodrug is metabolized in vivo to produce an agent as set forth herein are included within the scope of the claims. Prodrug forms of the herein described therapeutic agents, wherein the prodrug is metabolized in vivo to produce an agent as set forth herein are included within the scope of the claims. In some cases, some of the therapeutic agents described herein may be a prodrug for another derivative or active compound. In some embodiments described herein, hydrazones are metabolized in vivo to produce a therapeutic agent.

In certain embodiments, compositions provided herein include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, formulations described herein benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mMEDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

The pharmaceutical compositions described herein are formulated into any suitable dosage form, including but not limited to, aqueous oral dispersions, liquids, gels, syrups, elixirs, slurries, suspensions, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In one aspect, a therapeutic agent as discussed herein, e.g., therapeutic agent is formulated into a pharmaceutical composition suitable for intramuscular, subcutaneous, or intravenous injection. In one aspect, formulations suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles include water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. In some embodiments, formulations suitable for subcutaneous injection also contain additives such as preserving, wetting, emulsifying, and dispensing agents. Prevention of the growth of microorganisms can be ensured by various antibacterial and antifungal agents, such as para bens, chlorobutanol, phenol, sorbic acid, and the like. In some cases it is desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections or drips or infusions, a therapeutic agent described herein is formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art. For other parenteral injections, appropriate formulations include aqueous or nonaqueous solutions, preferably with physiologically compatible buffers or excipients. Such excipients are known.

Parenteral injections may involve bolus injection or continuous infusion. Formulations for injection may be presented in unit dosage form, e.g., in ampoules or in multi dose containers, with an added preservative. The pharmaceutical composition described herein may be in a form suitable for parenteral injection as a sterile suspensions, solutions or emulsions in oily or aqueous vehicles, and may contain formulatory agents such as suspending, stabilizing and/or dispersing agents. In one aspect, the active ingredient is in powder form for constitution with a suitable vehicle, e.g., sterile pyrogen-free water, before use.

For administration by inhalation, a therapeutic agent is formulated for use as an aerosol, a mist or a powder. Pharmaceutical compositions described herein are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the therapeutic agent described herein and a suitable powder base such as lactose or starch.

Representative intranasal formulations are described in, for example, U.S. Pat. Nos. 4,476,116, 5,116,817 and 6,391,452. Formulations that include a therapeutic agent are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are known to those skilled in the preparation of nasal dosage forms and some of these can be found in REMINGTON: THE SCIENCE AND PRACTICE OF PHARMACY, 21st edition, 2005. The choice of suitable carriers is dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present. Preferably, the nasal dosage form should be isotonic with nasal secretions.

Pharmaceutical preparations for oral use are obtained by mixing one or more solid excipient with one or more of the therapeutic agents described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active therapeutic agent doses.

In some embodiments, pharmaceutical formulations of a therapeutic agent are in the form of a capsules, including push fit capsules made of gelatin, as well as soft, sealed capsules made of gelatin and a plasticizer, such as glycerol or sorbitol. The push fit capsules contain the active ingredients in admixture with filler such as lactose, binders such as starches, and/or lubricants such as talc or magnesium stearate and, optionally, stabilizers. In soft capsules, the active therapeutic agent is dissolved or suspended in suitable liquids, such as fatty oils, liquid paraffin, or liquid polyethylene glycols. In some embodiments, stabilizers are added. A capsule may be prepared, for example, by placing the bulk blend of the formulation of the therapeutic agent inside of a capsule. In some embodiments, the formulations (non-aqueous suspensions and solutions) are placed in a soft gelatin capsule. In other embodiments, the formulations are placed in standard gelatin capsules or non-gelatin capsules such as capsules comprising HPMC. In other embodiments, the formulation is placed in a sprinkle capsule, wherein the capsule is swallowed whole or the capsule is opened and the contents sprinkled on food prior to eating.

All formulations for oral administration are in dosages suitable for such administration. In one aspect, solid oral dosage forms are prepared by mixing a therapeutic agent with one or more of the following: antioxidants, flavoring agents, and carrier materials such as binders, suspending agents, disintegration agents, filling agents, surfactants, solubilizers, stabilizers, lubricants, wetting agents, and diluents. In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder, a capsule, solid dispersion, solid solution, bioerodible dosage form, controlled release formulations, pulsatile release dosage forms, multiparticulate dosage forms, beads, pellets, granules. In other embodiments, the pharmaceutical formulation is in the form of a powder. Compressed tablets are solid dosage forms prepared by compacting the bulk blend of the formulations described above. In various embodiments, tablets will include one or more flavoring agents. In other embodiments, the tablets will include a film surrounding the final compressed tablet. In some embodiments, the film coating can provide a delayed release of a therapeutic agent from the formulation. In other embodiments, the film coating aids in patient compliance (e.g., Opadry® coatings or sugar coating). Film coatings including Opadry® typically range from about 1% to about 3% of the tablet weight. In some embodiments, solid dosage forms, e.g., tablets, effervescent tablets, and capsules, are prepared by mixing particles of a therapeutic agent with one or more pharmaceutical excipients to form a bulk blend composition. The bulk blend is readily subdivided into equally effective unit dosage forms, such as tablets, pills, and capsules. In some embodiments, the individual unit dosages include film coatings. These formulations are manufactured by conventional formulation techniques.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents. Exemplary useful microencapsulation materials include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG,HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to therapeutic agent the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Buccal formulations that include a therapeutic agent are administered using a variety of formulations known in the art. For example, such formulations include, but are not limited to, U.S. Pat. Nos. 4,229,447, 4,596,795, 4,755,386, and 5,739,136. In addition, the buccal dosage forms described herein can further include a bioerodible (hydrolysable) polymeric carrier that also serves to adhere the dosage form to the buccal mucosa. For buccal or sublingual administration, the compositions may take the form of tablets, lozenges, or gels formula ted in a conventional manner.

For intravenous injections, a therapeutic agent is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, a pharmaceutical composition described herein is in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an agent that modulates the activity of a carotid body in water soluble form. Additionally, suspensions of an agent that modulates the activity of a carotid body are optionally prepared as appropriate, e.g., oily injection suspensions.

Conventional formulation techniques include, e.g., one or a combination of methods: (1) dry mixing, (2) direct compression, (3) milling, (4) dry or non-aqueous granulation, (5) wet granulation, or (6) fusion. Other methods include, e.g., spray drying, pan coating, melt granulation, granulation, fluidized bed spray drying or coating (e.g., wurster coating), tangential coating, top spraying, tableting, extruding and the like.

Suitable carriers for use in the solid dosage forms described herein include, but are not limited to, acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose, hydroxypropylmethylcellulose acetate stearate, sucrose, microcrystalline cellulose, lactose, mannitol and the like.

Suitable filling agents for use in the solid dosage forms described herein include, but are not limited to, lactose, calcium carbonate, calcium phosphate, dibasic calcium phosphate, calcium sulfate, microcrystalline cellulose, cellulose powder, dextrose, dextrates, dextran, starches, pregelatinized starch, hydroxypropylmethycellulose (HPMC), hydroxypropylmethycellulose phthalate, hydroxypropylmethylcellulose acetate stearate (HPMCAS), sucrose, xylitol, lactitol, mannitol, sorbitol, sodium chloride, polyethylene glycol, and the like.

Suitable disintegrants for use in the solid dosage forms described herein include, but are not limited to, natural starch such as corn starch or potato starch, a pregelatinized starch, or sodium starch glycolate, a cellulose such as methylcrystalline cellulose, methylcellulose, microcrystalline cellulose, croscarmellose, or a cross-linked cellulose, such as cross-linked sodium carboxymethylcellulose, cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a gum such as agar, guar, locust bean, Karaya, pectin, or tragacanth, sodium starch glycolate, bentonite, sodium lauryl sulfate, sodium lauryl sulfate in combination starch, and the like.

Binders impart cohesiveness to solid oral dosage form formulations: for powder filled capsule formulation, they aid in plug formation that can be filled into soft or hard shell capsules and for tablet formulation, they ensure the tablet remaining intact after compression and help assure blend uniformity prior to a compression or fill step. Materials suitable for use as binders in the solid dosage forms described herein include, but are not limited to, carboxymethylcellulose, methylcellulose, hydroxypropylmethylcellulose, hydroxypropylmethylcellulose acetate stearate, hydroxyethylcellulose, hydroxypropylcellulose, ethylcellulose, and microcrystalline cellulose, microcrystalline dextrose, amylose, magnesium aluminum silicate, polysaccharide acids, bentonites, gelatin, polyvinylpyrrolidone/vinyl acetate copolymer, crospovidone, povidone, starch, pregelatinized starch, tragacanth, dextrin, a sugar, such as sucrose, glucose, dextrose, molasses, mannitol, sorbitol, xylitol, lactose, a natural or synthetic gum such as acacia, tragacanth, ghatti gum, mucilage of isapol husks, starch, polyvinylpyrrolidone, larch arabogalactan, polyethylene glycol, waxes, sodium alginate, and the like.

In general, binder levels of 20-70% are used in powder-filled gelatin capsule formulations. Binder usage level in tablet formulations varies whether direct compression, wet granulation, roller compaction, or usage of other excipients such as fillers which itself can act as moderate binder. Binder levels of up to 70% in tablet formulations is common.

Suitable lubricants or glidants for use in the solid dosage forms described herein include, but are not limited to, stearic acid, calcium hydroxide, talc, corn starch, sodium stearyl fumerate, alkali-metal and alkaline earth metal salts, such as aluminum, calcium, magnesium, zinc, stearic acid, sodium stearates, magnesium stearate, zinc stearate, waxes, Stearowet®, boric acid, sodium benzoate, sodium acetate, sodium chloride, leucine, a polyethylene glycol or a methoxypolyethylene glycol such as Carbowax™, PEG 4000, PEG 5000, PEG 6000, propylene glycol, sodium oleate, glyceryl behenate, glyceryl palmitostearate, glyceryl benzoate, magnesium or sodium lauryl sulfate, and the like.

Suitable diluents for use in the solid dosage forms described herein include, but are not limited to, sugars (including lactose, sucrose, and dextrose), polysaccharides (including dextrates and maltodextrin), polyols (including mannitol, xylitol, and sorbitol), cyclodextrins and the like.

Suitable wetting agents for use in the solid dosage forms described herein include, for example, oleic acid, glyceryl monostearate, sorbitan monooleate, sorbitan monolaurate, triethanolamine oleate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, vitamin E TPGS and the like.

Suitable surfactants for use in the solid dosage forms described herein include, for example, sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like.

Suitable suspending agents for use in the solid dosage forms described here include, but are not limited to, polyvinylpyrrolidone, e.g., polyvinylpyrrolidone K12, polyvinylpyrrolidone K17, polyvinylpyrrolidone K25, or polyvinylpyrrolidone K30, polyethylene glycol, e.g., the polyethylene glycol can have a molecular weight of about 300 to about 6000, or about 3350 to about 4000, or about 7000 to about 5400, vinyl pyrrolidone/vinyl acetate copolymer (S630), sodium carboxymethylcellulose, methylcellulose, hydroxy-propylmethylcellulose, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Suitable antioxidants for use in the solid dosage forms described herein include, for example, e.g., butylated hydroxytoluene (BHT), sodium ascorbate, and tocopherol.

It should be appreciated that there is considerable overlap between additives used in the solid dosage forms described herein. Thus, the above-listed additives should be taken as merely exemplary, and not limiting, of the types of additives that can be included in solid dosage forms of the pharmaceutical compositions described herein. The amounts of such additives can be readily determined by one skilled in the art, according to the particular properties desired.

In various embodiments, the particles of a therapeutic agents and one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the formulation into the gastrointestinal fluid.

In other embodiments, a powder including a therapeutic agent is formulated to include one or more pharmaceutical excipients and flavors. Such a powder is prepared, for example, by mixing the therapeutic agent and optional pharmaceutical excipients to form a bulk blend composition. Additional embodiments also include a suspending agent and/or a wetting agent. This bulk blend is uniformly subdivided into unit dosage packaging or multi-dosage packaging units.

In still other embodiments, effervescent powders are also prepared. Effervescent salts have been used to disperse medicines in water for oral administration.

In some embodiments, the pharmaceutical dosage forms are formulated to provide a controlled release of a therapeutic agent. Controlled release refers to the release of the therapeutic agent from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates can provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In some embodiments, the solid dosage forms described herein are formulated as enteric coated delayed release oral dosage forms, i.e., as an oral dosage form of a pharmaceutical composition as described herein which utilizes an enteric coating to affect release in the small intestine or large intestine. In one aspect, the enteric coated dosage form is a compressed or molded or extruded tablet/mold (coated or uncoated) containing granules, powder, pellets, beads or particles of the active ingredient and/or other composition components, which are themselves coated or uncoated. In one aspect, the enteric coated oral dosage form is in the form of a capsule containing pellets, beads or granules, which include a therapeutic agent that are coated or uncoated.

Any coatings should be applied to a sufficient thickness such that the entire coating does not dissolve in the gastrointestinal fluids at pH below about 5, but does dissolve at pH about 5 and above. Coatings are typically selected from any of the following: Shellac-this coating dissolves in media of pH>7; Acrylic polymers-examples of suitable acrylic polymers include methacrylic acid copolymers and ammonium methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for colonic targeting. The Eudragit series E dissolve in the stomach. The Eudragit series L, L-30D and S are insoluble in stomach and dissolve in the intestine; Poly Vinyl Acetate Phthalate (PVAP)-PVAP dissolves in pH >5, and it is much less permeable to water vapor and gastric fluids. Conventional coating techniques such as spray or pan coating are employed to apply coatings. The coating thickness must be sufficient to ensure that the oral dosage form remains intact until the desired site of topical delivery in the intestinal tract is reached.

In other embodiments, the formulations described herein are delivered using a pulsatile dosage form. A pulsatile dosage form is capable of providing one or more immediate release pulses at predetermined time points after a controlled lag time or at specific sites. Exemplary pulsatile dosage forms and methods of their manufacture are disclosed in U.S. Pat. Nos. 5,011,692, 5,017,381, 5,229,135, 5,840,329 and 5,837,284. In one embodiment, the pulsatile dosage form includes at least two groups of particles, (i.e. multiparticulate) each containing the formulation described herein. The first group of particles provides a substantially immediate dose of a therapeutic agent upon ingestion by a mammal. The first group of particles can be either uncoated or include a coating and/or sealant. In one aspect, the second group of particles comprises coated particles. The coating on the second group of particles provides a delay of from about 2 hours to about 7 hours following ingestion before release of the second dose. Suitable coatings for pharmaceutical compositions are described herein or known in the art.

In some embodiments, pharmaceutical formulations are provided that include particles of a therapeutic agent and at least one dispersing agent or suspending agent for oral administration to a subject. The formulations may be a powder and/or granules for suspension, and upon admixture with water, a substantially uniform suspension is obtained.

In some embodiments, particles formulated for controlled release are incorporated in a gel or a patch or a wound dressing.

In one aspect, liquid formulation dosage forms for oral administration and/or for topical administration as a wash are in the form of aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to the particles of a therapeutic agent, the liquid dosage forms include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions can further include a crystalline inhibitor.

In some embodiments, the liquid formulations also include inert diluents commonly used in the art, such as water or other solvents, solubilizing agents, and emulsifiers. Exemplary emulsifiers are ethyl alcohol, isopropyl alcohol, ethyl carbonate, ethyl acetate, benzyl alcohol, benzyl benzoate, propyleneglycol, 1,3-butyleneglycol, dimethylformamide, sodium lauryl sulfate, sodium docusate, cholesterol, cholesterol esters, taurocholic acid, phosphatidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In one embodiment, the aqueous suspensions and dispersions described herein remain in a homogenous state, as defined in The USP Pharmacists' Pharmacopeia (2005 edition, chapter 905), for at least 4 hours. In one embodiment, an aqueous suspension is re-suspended into a homogenous suspension by physical agitation lasting less than 1 minute. In still another embodiment, no agitation is necessary to maintain a homogeneous aqueous dispersion.

Examples of disintegrating agents for use in the aqueous suspensions and dispersions include, but are not limited to, a starch, e.g., a natural starch such as corn starch or potato starch, a pregelatinized starch, or sodium starch glycolate; a cellulose such as methylcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein include, for example, hydrophilic polymers, electrolytes, Tween® 60 or 80, PEG, polyvinylpyrrolidone, and the carbohydrate-based dispersing agents such as, for example, hydroxypropylcellulose and hydroxypropyl cellulose ethers, hydroxypropyl methylcellulose and hydroxypropyl methylcellulose ethers, carboxymethylcellulose sodium, methylcellulose, hydroxyethylcellulose, hydroxypropylmethyl-cellulose phthalate, hydroxypropylmethyl-cellulose acetate stearate, noncrystalline cellulose, magnesium aluminum silicate, triethanolamine, polyvinyl alcohol (PVA), polyvinylpyrrolidone/vinyl acetate copolymer, 4-(1,1,3,3-tetramethylbutyl)-phenol polymer with ethylene oxide and formaldehyde (also known as tyloxapol), poloxamers; and poloxamines. In other embodiments, the dispersing agent is selected from a group not comprising one of the following agents: hydrophilic polymers; electrolytes; Tween® 60 or 80; PEG; polyvinylpyrrolidone (PVP); hydroxypropylcellulose and hydroxypropyl cellulose ethers; hydroxypropyl methylcellulose and hydroxypropyl methylcellulose ethers; carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers; or poloxamines.

Wetting agents suitable for the aqueous suspensions and dispersions described herein include, but are not limited to, cetyl alcohol, glycerol monostearate, polyoxyethylene sorbitan fatty acid esters (e.g., the commercially available Tweens® such as e.g., Tween 20® and Tween 80®, and polyethylene glycols, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphatidylcholine and the like.

Suitable preservatives for the aqueous suspensions or dispersions described herein include, for example, potassium sorbate, parabens (e.g., methylparaben and propylparaben), benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Suitable viscosity enhancing agents for the aqueous suspensions or dispersions described herein include, but are not limited to, methyl cellulose, xanthan gum, carboxymethyl cellulose, hydroxypropyl cellulose, hydroxypropylmethyl cellulose, Plasdon® S-630, carbomer, polyvinyl alcohol, alginates, acacia, chitosans and combinations thereof. The concentration of the viscosity enhancing agent will depend upon the agent selected and the viscosity desired.

Examples of sweetening agents suitable for the aqueous suspensions or dispersions described herein include, for example, acacia syrup, acesulfame K, alitame, aspartame, chocolate, cinnamon, citrus, cocoa, cyclamate, dextrose, fructose, ginger, glycyrrhetinate, glycyrrhiza (licorice) syrup, monoammonium glyrrhizinate (MagnaSweet®), malitol, mannitol, menthol, neohesperidine DC, neotame, Prosweet® Powder, saccharin, sorbitol, stevia, sucralose, sucrose, sodium saccharin, saccharin, aspartame, acesulfame potassium, mannitol, sucralose, tagatose, thaumatin, vanilla, xylitol, or any combination thereof.

In some embodiments, a therapeutic agent is prepared as transdermal dosage form. In some embodiments, the transdermal formulations described herein include at least three components: (1) a therapeutic agent; (2) a penetration enhancer; and (3) an optional aqueous adjuvant. In some embodiments the transdermal formulations include additional components such as, but not limited to, gelling agents, creams and ointment bases, and the like. In some embodiments, the transdermal formulation is presented as a patch or a wound dressing. In some embodiments, the transdermal formulation further include a woven or non-woven backing material to enhance absorption and prevent the removal of the transdermal formulation from the skin. In other embodiments, the transdermal formulations described herein can maintain a saturated or supersaturated state to promote diffusion into the skin.

In one aspect, formulations suitable for transdermal administration of a therapeutic agent described herein employ transdermal delivery devices and transdermal delivery patches and can be lipophilic emulsions or buffered, aqueous solutions, dissolved and/or dispersed in a polymer or an adhesive. In one aspect, such patches are constructed for continuous, pulsatile, or on demand delivery of pharmaceutical agents. Still further, transdermal delivery of the therapeutic agents described herein can be accomplished by means of iontophoretic patches and the like. In one aspect, transdermal patches provide controlled delivery of a therapeutic agent. In one aspect, transdermal devices are in the form of a bandage comprising a backing member, a reservoir containing the therapeutic agent optionally with carriers, optionally a rate controlling barrier to deliver the therapeutic agent to the skin of the host at a controlled and predetermined rate over a prolonged period of time, and means to secure the device to the skin.

In further embodiments, topical formulations include gel formulations (e.g., gel patches which adhere to the skin). In some of such embodiments, a gel composition includes any polymer that forms a gel upon contact with the body (e.g., gel formulations comprising hyaluronic acid, pluronic polymers, poly(lactic-co-glycolic acid (PLGA)-based polymers or the like). In some forms of the compositions, the formulation comprises a low-melting wax such as, but not limited to, a mixture of fatty acid glycerides, optionally in combination with cocoa butter which is first melted. Optionally, the formulations further comprise a moisturizing agent.

In certain embodiments, delivery systems for pharmaceutical therapeutic agents may be employed, such as, for example, liposomes and emulsions. In certain embodiments, compositions provided herein can also include an mucoadhesive polymer, selected from among, for example, carboxymethylcellulose, carbomer (acrylic acid polymer), poly(methylmethacrylate), polyacrylamide, polycarbophil, acrylic acid/butyl acrylate copolymer, sodium alginate and dextran.

In some embodiments, a therapeutic agent described herein may be administered topically and can be formulated into a variety of topically administrable compositions, such as solutions, suspensions, lotions, gels, pastes, medicated sticks, balms, creams or ointments. Such pharmaceutical therapeutic agents can contain solubilizers, stabilizers, tonicity enhancing agents, buffers and preservatives.

### Kits

Disclosed herein, are kits useful for to detect the genotypes and/or biomarkers disclosed herein. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. Thus, in some embodiments the kit contains a composition including of the pharmaceutical composition, for the treatment of IBD. In other embodiments, the kits contains all of the components necessary and/or sufficient to perform an assay for detecting and measuring IBD markers, including all controls, directions for performing assays, and any necessary software for analysis and presentation of results.

In some instances, the kits described herein comprise components for detecting the presence, absence, and/or quantity of a target nucleic acid and/or protein described herein. In some embodiments, the kit further comprises components for detecting the presence, absence, and/or quantity of a serological marker described herein. In some embodiments, the kit comprises the compositions (e.g., primers, probes, antibodies) described herein. The disclosure provides kits suitable for assays such as enzyme-linked immunosorbent assay (ELISA), single-molecular array (Simoa), PCR, and qPCR. The exact nature of the components configured in the kit depends on its intended purpose.

In some embodiments, the kits described herein are configured for the purpose of treating and/or characterizing a disease or condition (e.g., Crohn's disease), or subclinical phenotype thereof (e.g., stricturing, penetrating, or stricturing and penetrating disease phenotypes) in a subject. In some embodiments, the kits described herein are configured for the purpose of identifying a subject suitable for treatment with a therapeutic agent (e.g., anti-TL1A antibody or acetate inhibitor). In some embodiments, the kit is configured particularly for the purpose of treating mammalian subjects. In some embodiments, the kit is configured particularly for the purpose of treating human subjects. In further embodiments, the kit is configured for veterinary applications, treating subjects such as, but not limited to, farm animals, domestic animals, and laboratory animals. In some embodiments, the kit is configured to select a subject for a therapeutic agent, such as those disclosed herein. In some embodiments, the kit is configured to select a subject for treatment with a therapeutic agent disclosed herein.

Instructions for use may be included in the kit. Optionally, the kit also contains other useful components, such as, diluents, buffers, pharmaceutically acceptable carriers, syringes, catheters, applicators, pipetting or measuring tools, bandaging materials or other useful paraphernalia. The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components. Thus, for example, a package can be a glass vial or prefilled syringes used to contain suitable quantities of the pharmaceutical composition. The packaging material has an external label which indicates the contents and/or purpose of the kit and its components.

### Systems

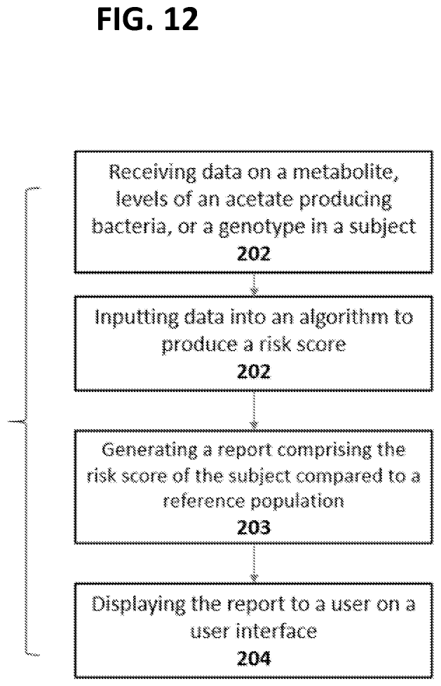

Disclosed herein are systems for identifying a subject as likely to develop ileitis, intestinal fibrosis, or colitis. In some embodiments, the systems described herein comprise kits and compositions for detecting the metabolites, acetate-producing bacteria, or genotypes described herein in a biological sample of a subject. The system may comprise a computer system for implementing one or more methods of the disclosure, such as for example, receiving data on the metabolites, acetate-producing bacteria, or genotypes of a subject 201, inputting the genotype data into an algorithm to produce a risk score 202, and generating a report comprising the risk score of the subject compared to a reference population 203, and displaying the report to a user on a graphical user interface 204, as shown in FIG. 12. A “risk profile” as used herein refers to a profile of expression of one or more features described herein in a subject that is detected in a biological sample obtained from the subject. In some embodiments, a risk profile comprises a positive, a negative, or an indeterminate result (e.g., therapeutic response to treatment with an inhibitor of acetate producing bacteria).

### Computer Systems

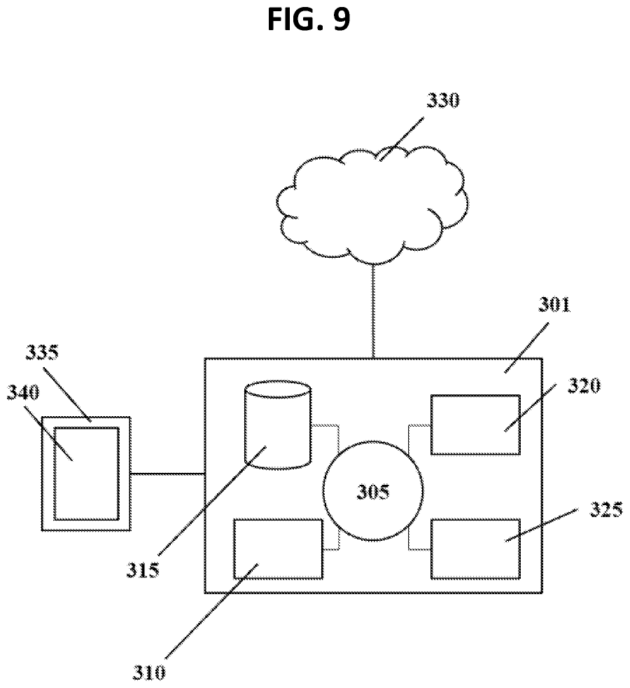

FIG. 9 shows a computer system 301 that is programmed or otherwise configured to generate a risk profile for a subject in need thereof. The computer system 301 can regulate various aspects of producing the risk profile (e.g., receiving data, generating a report with the risk profile of the biological sample, and displaying the report to a user), of the present disclosure, such as, for example, by including permissions or encryption of metabolite, levels of acetate producing bacteria, or genotype data and/or genetic risk profile of the subject to ensure patient privacy. In some embodiments the computer system selects one or more metabolites. In some embodiments, the metabolite is acetate. In some embodiments, the computer system selects one or more genetic variants. In some embodiments, the one of more genetic variants comprise one or more genotypes of the subject or a predetermined genetic variant in a linkage disequilibrium (LD) therewith. In some embodiments, the one or more genetic variants comprises rs3810936, rs6478108, rs6478 109, rs7848647, or rs7869487. In some embodiments, the computer system calculates a genetic risk score for the subject, based, at least in part on the one or more genetic variants.

In some embodiments, the computer system predicts a subject that is likely to develop ileitis, intestinal fibrosis or colitis. In some embodiments, the computer system predicts a subject that is likely to develop ileitis, intestinal fibrosis or colitis based on measuring an increase in an amount of the acetate, a metabolite thereof, or an acetate producing bacteria. In some embodiments, the computer system predicts a subject that is likely to develop ileitis, intestinal fibrosis or colitis based on detection of hyperplasia or morphology of a population of Paneth cells. In some embodiments, the computer system predicts a subject that is likely to develop ileitis, intestinal fibrosis or colitis based on a measurement of at least one metabolite. In some embodiments, the computer system selects a subject that is likely to develop ileitis, intestinal fibrosis or colitis for treatment to prevent the formation of ileitis, intestinal fibrosis or colitis.

In some embodiments, the computer system selects a subject for treatment with an active agent to reduce the amount of acetate producing bacteria in the subject. In some embodiments, the computer system selects a subject for treatment with a fecal microbiota transplant. In some embodiments, the computer system selects a subject for treatment with a probiotic. In some embodiments, the computer system selects a subject for treatment with a chelating agent. In some embodiments, the computer system selects a subject for treatment with an inhibitor of acetate or acetate producing bacteria as described herein. In some embodiments, the computer system selects a subject for treatment with a TL1a inhibitor described herein.

The computer system 301 can be an electronic device of a user or a computer system that is remotely located with respect to the electronic device. The electronic device can be a mobile electronic device, such as a mobile electronic device belonging to a physician.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, a report comprising the genetic risk profile of the subject or other relevant clinical information for purposes of informing a selection of a therapeutic agent (e.g., anti-TL1A antibody) to treat a disease or condition of the subject described herein. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, perform: (a) receiving genotype data of a subject, (b) determining whether the genotypes are heterozygous or homozygous for nine polymorphisms, (c) generating an outcome using predetermined parameters, and (d) displaying the outcome to a user (e.g., physician) on a user interface of an electronic device. In some embodiments, the outcome is positive, negative or indeterminant. In some embodiments, the predetermined parameters are genotype combinations known to be predictive of a therapeutic response to a treatment, such as with an inhibitor of acetate producing bacteria.

### Web Application

In some embodiments, the computer system comprises software for a web application. In light of the disclosure provided herein, in some embodiments, a web application may utilize one or more software frameworks and one or more database systems. A web application, for example, is created upon a software framework such as Microsoft®.NET or Ruby on Rails (RoR). A web application, In some embodiments, utilizes one or more database systems including, by way of non-limiting examples, relational, non-relational, feature oriented, associative, and XML database systems. Suitable relational database systems include, by way of non-limiting examples, Microsoft® SQL Server, my SQL™, and Oracle®. In some embodiments, a web application may be written in one or more versions of one or more languages. In some embodiments, a web application is written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). A web application may integrate enterprise server products such as IBM® Lotus Domino®. A web application may include a media player element. A media player element may utilize one or more of many suitable multimedia technologies including, by way of non-limiting examples, Adobe® Flash®, HTML 5, Apple® QuickTime®, Microsoft® Silverlight®, Java™, and Unity®.

### Mobile Application

In some embodiments, the computer system comprises software for a mobile application. The mobile application may be provided to a mobile digital processing device at the time it is manufactured. The mobile application may be provided to a mobile digital processing device via the computer network described herein.

A mobile application is created by using hardware, languages, and development environments. In some embodiments, the mobile applications may be written in several languages. Suitable programming languages include, by way of non-limiting examples, C, C++, C#, Featureive-C, Java™, Javascript, Pascal, Feature Pascal, Python™, Ruby, VB.NET, WML, and XHTML/HTML with or without CSS, or combinations thereof.

Suitable mobile application development environments are available from several sources. Commercially available development environments include, by way of non-limiting examples, Airplay SDK, alcheMo, Appcelerator®, Celsius, Bedrock, Flash Lite, .NET Compact Framework, Rhomobile, and WorkLight Mobile Platform. Other development environments may be available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, the computer system comprises software a standalone application, which is a program that may be run as an independent computer process, not an add-on to an existing process, e.g., not a plug-in. In some embodiments, standalone applications are sometimes compiled. In some embodiments, a compiler is a computer program(s) that transforms source code written in a programming language into binary feature code such as assembly language or machine code. Suitable compiled programming languages include, by way of non-limiting examples, C, C++, Featureive-C, COBOL, Delphi, Eiffel, Java™, Lisp, Python™, Visual Basic, and VB.NET, or combinations thereof. Compilation may be often performed, at least in part, to create an executable program. In some embodiments, a computer program includes one or more executable complied applications.

### Web Browser Plug-In

In some embodiments, the computer system comprises software that comprises a web browser plug-in. In computing, a plug-in, In some embodiments, is one or more software components that add specific functionality to a larger software application. Makers of software applications may support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Examples of web browser plug-ins include, without limitations, Adobe® Flash® Player, Microsoft® Silverlight®, and Apple® QuickTime®. The toolbar may comprise one or more web browser extensions, add-ins, or add-ons. The toolbar may comprise one or more explorer bars, tool bands, or desk bands.

In view of the disclosure provided herein, plug-in frameworks are available that enable development of plug-ins in various programming languages, include, by way of non-limiting examples, C++, Delphi, Java™, PHP, Python™, and VB.NET, or combinations thereof.

In some embodiments, Web browsers (also called Internet browsers) are software applications, designed for use with network-connected digital processing devices, for retrieving, presenting, and traversing information resources on the World Wide Web. Suitable web browsers include, by way of non-limiting examples, Microsoft® Internet Explorer®, Mozilla Firefox® Google® Chrome, Apple® Safari®, Opera Software® Opera®, and KDE Konqueror. The web browser, In some embodiments, is a mobile web browser. Mobile web browsers (also called microbrowsers, mini-browsers, and wireless browsers) may be designed for use on mobile digital processing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

The medium, method, and system disclosed herein comprise one or more softwares, servers, and database modules, or use of the same. In view of the disclosure provided herein, software modules may be created by using machines, software, and languages. The software modules disclosed herein may be implemented in a multitude of ways. In some embodiments, a software module comprises a file, a section of code, a programming feature, a programming structure, or combinations thereof. A software module may comprise a plurality of files, a plurality of sections of code, a plurality of programming features, a plurality of programming structures, or combinations thereof. By way of non-limiting examples, the one or more software modules comprise a web application, a mobile application, and/or a standalone application. Software modules may be in one computer program or application. Software modules may be in more than one computer program or application. Software modules may be hosted on one machine. Software modules may be hosted on more than one machine. Software modules may be hosted on cloud computing platforms. Software modules may be hosted on one or more machines in one location. Software modules may be hosted on one or more machines in more than one location.

### Databases

The medium, method, and system disclosed herein comprise one or more databases, or use of the same. In view of the disclosure provided herein, some databases are suitable for storage and retrieval of geologic profile, operator activities, division of interest, and/or contact information of royalty owners may be used. Suitable databases include, by way of non-limiting examples, relational databases, non-relational databases, feature oriented databases, feature databases, entity-relationship model databases, associative databases, and XML databases. In some embodiments, a database is internet-based. In some embodiments, a database is web-based. In some embodiments, a database is cloud computing-based. A database may be based on one or more local computer storage devices.

### Data Transmission

The subject matter described herein, including methods for producing a genetic risk profile are configured to be performed in one or more facilities at one or more locations. Facility locations are not limited by country and include any country or territory. In some embodiments, one or more steps are performed in a different country than another step of the method. In some embodiments, one or more steps for obtaining a sample are performed in a different country than one or more steps for detecting the presence or absence of a genotype in a biological sample. In some embodiments, one or more method steps involving a computer system are performed in a different country than another step of the methods provided herein. In some embodiments, data processing and analyses are performed in a different country or location than one or more steps of the methods described herein. In some embodiments, one or more articles, products, or data are transferred from one or more of the facilities to one or more different facilities for analysis or further analysis. An article includes, but is not limited to, one or more components obtained from a subject, e.g., processed cellular material. Processed cellular material includes, but is not limited to, cDNA reverse transcribed from RNA, amplified RNA, amplified cDNA, sequenced DNA, isolated and/or purified RNA, isolated and/or purified DNA, and isolated and/or purified polypeptide. Data includes, but is not limited to, information regarding the stratification of a subject, and any data produced by the methods disclosed herein. In some embodiments of the methods and systems described herein, the analysis is performed and a subsequent data transmission step will convey or transmit the results of the analysis.

In some embodiments, any step of any method described herein is performed by a software program or module on a computer. In additional or further embodiments, data from any step of any method described herein is transferred to and from facilities located within the same or different countries, including analysis performed in one facility in a particular location and the data shipped to another location or directly to an individual in the same or a different country. In additional or further embodiments, data from any step of any method described herein is transferred to and/or received from a facility located within the same or different countries, including analysis of a data input, such as genetic or processed cellular material, performed in one facility in a particular location and corresponding data transmitted to another location, or directly to an individual, such as data related to the diagnosis, prognosis, responsiveness to therapy (e.g., anti-TL1A therapy), or the like, in the same or different location or country.

### Business Methods Utilizing a Computer

The methods described herein may utilize one or more computers. The computer may be used for managing customer and biological sample information such as sample or customer tracking, database management, analyzing molecular profiling data, analyzing cytological data, storing data, billing, marketing, reporting results, storing results, or a combination thereof. The computer may include a monitor or other user interface for displaying data, results, billing information, marketing information (e.g. demographics), customer information, or sample information. The computer may also include means for data or information input. The computer may include a processing unit and fixed or removable media or a combination thereof. The computer may be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer may be connected to a server or other communication device for relaying information from a user to the computer or from the computer to a user. In some cases, the user may store data or information obtained from the computer through a communication medium on media, such as removable media. It is envisioned that data relating to the methods can be transmitted over such networks or connections for reception and/or review by a party. The receiving party can be but is not limited to an individual, a health care provider (e.g., physician) or a health care manager. In one embodiment, a computer-readable medium includes a medium suitable for transmission of a result of an analysis of a biological sample, such as exosome bio-signatures. The medium can include a result regarding an exosome bio-signature of a subject, wherein such a result is derived using the methods described herein.

The entity obtaining a report with the genetic risk profile may enter biological sample information into a database for the purpose of one or more of the following: inventory tracking, assay result tracking, order tracking, customer management, customer service, billing, and sales. Sample information may include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history may include but is not limited to: age of the sample, type of sample, method of acquisition, method of storage, or method of transport.

The database may be accessible by a customer, medical professional, insurance provider, or other third party. Database access may take the form of electronic communication such as a computer or telephone. The database may be accessed through an intermediary such as a customer service representative, business representative, consultant, independent testing center, or medical professional. The availability or degree of database access or sample information, such as assay results, may change upon payment of a fee for products and services rendered or to be rendered. The degree of database access or sample information may be restricted to comply with generally accepted or legal requirements for patient or customer confidentiality.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease. The term “in vivo” is used to describe an event that takes place in a subject's body.

The term “ex vivo” is used to describe an event that takes place outside of a subject's body. An ex vivo assay is not performed on a subject. Rather, it is performed up on a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215 (3): 403-10; Nucleic Acids Res. 1997 Sep. 1; 25 (17): 3389-402). Percent homology of sequences can be determined using the most recent version of BLAST, as of the filing date of this application. Percent identity of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level. An increase can be an absolute amount (e.g., level of protein expression), or a rate of production (e.g., rate of protein expression between two points in time). In the context of a marker or symptom, by these terms is meant a statistically significant increase in such level. The increase can be, for example, at least 10%, at least 20%, at least 30%, at least 40% or more of a level accepted as within the range of normal for an individual without a given disease

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level. A decrease can be an absolute amount (e.g., level of protein expression), or a rate of production (e.g., rate of protein expression between two points in time). In the context of a marker or symptom, by these terms is meant a statistically significant decrease in such level. The decrease can be, for example, at least 10%, at least 20%, at least 30%, at least 40% or more of a level accepted as within the range of normal for an individual without a given disease.

The terms “subject” encompass mammals. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In some embodiments, a human subject is a “patient,” which as used herein, refers to a subject who has or may be diagnosed with a disease or condition disclosed herein.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence. A “genetic locus” referred to herein, is a particular location within a gene.

The term, “genotype” as disclosed herein, refers to the chemical composition of a polynucleotide sequence within the genome of an individual. In some embodiments, the genotype comprises a single nucleotide polymorphism (SNP) or and indel (insertion or deletion, of a nucleobase within a polynucleotide sequence). In some embodiments, a genotype for a particular SNP, or indel is heterozygous. In some embodiments, a genotype for a particular SNP, or indel is homozygous.

A “polymorphism” as used herein refers to an aberration in (e.g., a mutation), or of (e.g., insertion/deletion), a nucleic acid sequence, as compared to the nucleic acid sequence in a reference population. In some embodiments, the polymorphism is common in the reference population. In some embodiments, the polymorphism is rare in the reference population. In some embodiments, the polymorphism is a single nucleotide polymorphism.

The term, “single nucleotide polymorphism” or SNP as disclosed herein, refers to a variation in a single nucleotide within a poly nucleotide sequence. The term should not be interpreted as placing a restriction on a frequency of the SNP in a given population. The variation of an SNP may have multiple different forms. A single form of an SNP is referred to as an “allele.” An SNP can be mono-, bi-, tri, or tetra-allelic. A SNP may include a “risk allele,” a “protective allele,” or neither. By way of example, a reference polynucleotide sequence reading 5′ to 3′ is TTACG. A SNP at allele position 3 (of 5′-TTACG-3′) comprise a substitution of the reference allele, “A” to a non-reference allele, “C.” If the “C” allele of the SNP is associated with an increased probability of developing a phenotypic trait, the allele is considered a “risk” allele. However, the same SNP may also comprise a substitution of the “A” allele to a “T” allele at position 3. If the T allele of the SNP is associated with a decreased probability of developing a phenotypic trait, the allele is considered a “protective” allele. The SNP may be observed in at least 1% of a given population. In some embodiments, the SNP is represented by an “rs” number, which refers to the accession of reference cluster of one more submitted SNPs in the db SNP bioinformatics database as of the filing date of this patent application, and which is included within a sequence that comprises the total number of nucleobases from 5′ to 3′. In some embodiments, a SNP may be further defined by the position of the SNP (nucleobase) within the dbSNP sequence, the position of which is always with reference to 5′ length of the sequence plus 1. In some embodiments, a SNP is defined as the genomic position in a reference genome and the allele change (e.g. chromosome 7 at position 234, 123,567 from G allele to A allele in the reference human genome build 37). In some embodiments, the SNV is defined as the genomic position identified with [brackets] or an “N” in a sequence disclosed herein.

The term, “indel,” as disclosed herein, refers to an insertion, or a deletion, of a nucleobase within a polynucleotide sequence. An indel can be mono-, bi-, tri, or tetra-allelic. An indel may be “risk,” a “protective,” or neither, for a phenotypic trait. In some embodiments, the indel is represented by an “rs” number, which refers to the accession of reference cluster of one more submitted indels in the db SNP bioinformatics database as of the filing date of this patent application, and which is included in a sequence that comprises the total number of nucleobases from 5′ to 3′. In some embodiments, an indel may be further defined by the position of the insertion/deletion within the dbSNP sequence, the position of which is always with reference to the 5′ length of the sequence plus 1. In some embodiments, an indel is defined as the genomic position in a reference genome and the allele change. In some embodiments, the indel is defined as the genomic position identified with [brackets] or an “N” in a sequence disclosed herein.

“Haplotype” as used herein, encompasses a group of one or more genotypes, which tend to be inherited together in a reference population. In some embodiments, a haplotype comprises particular polymorphism or another polymorphism in linkage disequilibrium (LD) therewith.

“Linkage disequilibrium,” or “LD,” as used herein refers to the non-random association of alleles or indels in different gene loci in a given population. LD may be defined by a D′ value corresponding to the difference between an observed and expected allele or indel frequencies in the population (D-Pab-PaPb), which is scaled by the theoretical maximum value of D. LD may be defined by an r2 value corresponding to the difference between an observed and expected unit of risk frequencies in the population (D=Pab-PaPb), which is scaled by the individual frequencies of the different loci. In some embodiments, D′ comprises at least 0.20. In some embodiments, r2 comprises at least 0.70.

The term “medically refractory,” or “refractory,” as used herein, refers to the failure of a first-line or non-curative treatment to induce remission of a disease. In some embodiments, the disease comprises an inflammatory disease disclosed herein. A non-limiting example of refractory inflammatory disease includes refractory Crohn's disease, and refractory ulcerative colitis (e.g., mrUC).

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21 st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, oral, injection, aerosol, parenteral, and topical administration.

The term “inflammatory bowel disease” or “IBD” as used herein refers to gastrointestinal disorders of the gastrointestinal tract. Non-limiting examples of IBD include, Crohn's disease (CD), ulcerative colitis (UC), indeterminate colitis (IC), microscopic colitis, diversion colitis, Behcet's disease, and other inconclusive forms of IBD. In some embodiments, IBD comprises fibrosis, fibrostenosis, stricturing and/or penetrating disease, obstructive disease, or a disease that is refractory (e.g., mrUC, refractory CD), perianal CD, or other complicated forms of IBD.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. In various embodiments, the sample comprises tissue from the large and/or small intestine. In various embodiments, the large intestine sample comprises the cecum, colon (the ascending colon, the transverse colon, the descending colon, and the sigmoid colon), rectum and/or the anal canal. In some embodiments, the small intestine sample comprises the duodenum, jejunum, and/or the ileum. Alternatively, a sample can be obtained through primary patient derived cell lines, or archived patient samples in the form of preserved samples, or fresh frozen samples.

The term “biomarker” comprises a measurable substance in a subject whose presence, level, or activity, is indicative of a phenomenon (e.g., phenotypic expression or activity; disease, condition, subclinical phenotype of a disease or condition, infection; or environmental stimuli). In some embodiments, a biomarker comprises a gene, gene expression product (e.g., RNA or protein), or a cell-type (e.g., immune cell).

The term “serological marker,” as used herein refers to a type of biomarker representing an antigenic response in a subject that may be detected in the serum of the subject. In some embodiments, a serological comprises an antibody against various fungal antigens. Non-limiting examples of a serological marker comprise anti-Saccharomyces cerevisiae antibody (ASCA), an anti-neutrophil cytoplasmic antibody (ANCA), E. coli outer membrane porin protein C (OmpC), anti-Malassezia restricta antibody, anti-Malassezia pachydermatis antibody, anti-Malassezia furfur antibody, anti-Malassezia globasa antibody, anti-Cladosporium albicans antibody, anti-laminaribiose antibody (ALCA), anti-chitobioside antibody (ACCA), anti-laminarin antibody, anti-chitin antibody, pANCA antibody, anti-12 antibody, and anti-Cbir1 flagellin antibody.

The term “microbiome” and its variation used herein describe the populations and interactions of the bacteria, fungi, protists, and virus that align the gastrointestinal tract of a subject. A subject afflicted with IBD may possess presence, absence, excess, diminished, or a combination thereof of a microbiome s compared to a healthy subject.

The terms “non-response,” or “loss-of-response,” as used herein, refer to phenomena in which a subject or a patient does not respond to the induction of a standard treatment (e.g., anti-TNF therapy), or experiences a loss of response to the standard treatment after a successful induction of the therapy. The induction of the standard treatment may include 1, 2, 3, 4, or 5, doses of the therapy. A “successful induction” of the therapy may be an initial therapeutic response or benefit provided by the therapy. The loss of response may be characterized by a reappearance of symptoms consistent with a flare after a successful induction of the therapy.

The section headings used herein are for organizational purposes only and are not to be construed as limiting the subject matter described.

### EXAMPLES

The following illustrative examples are representative of embodiments of the software applications, systems, and methods described herein and are not meant to be limiting in any way.

### Example 1: Paneth Cell Abnormalities Precede the Development of Small Intestinal Inflammation in Mice Overexpressing TL1A

It was previously demonstrated that mice over-expressing TL1A (Tl1a-tg) developed spontaneous ileitis over time. Histological examinations of tl1a-tg mice revealed an increased histoscore and increased numbers of Goblet and Paneth cells when compared to the histological examinations of wild-type (WT) mice. In this example, the molecular mechanisms that lead to Paneth cell hyperplasia in Tl1a-tg mice were explored.

Mice: Age-matched and gender-matched mice on the C57BL/6J background were used. LCK-CD2-Tl1a-GFP (Tl1a-tg) mice and littermate wild-type (WT) mice were used.

Histology Scoring: Ileal tissues were collected 1-2 cm long near cecum, paraffin-embedded and stained with H&E for histological analysis. Severity of ileal inflammation was evaluated in a blinded manner according to previously described criteria by two trained animal pathologists. Spontaneous ileitis was evaluated by scoring the degree of inflammation, extent of involvement, crypt damage and villus changes as described. GF mice were evaluated using a scoring criterion.

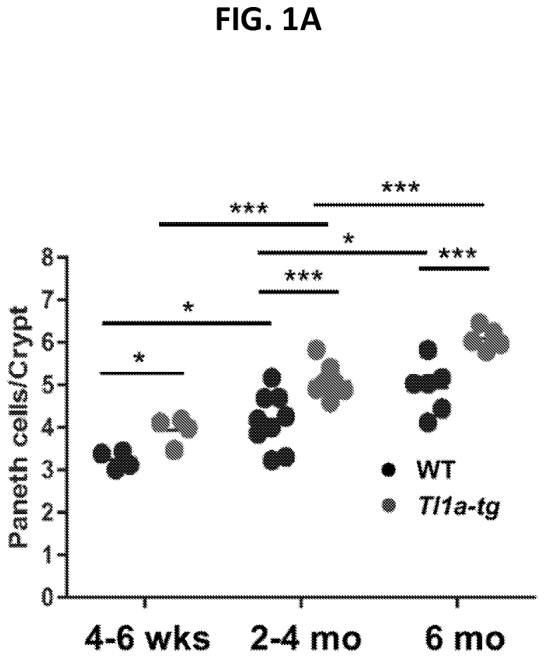

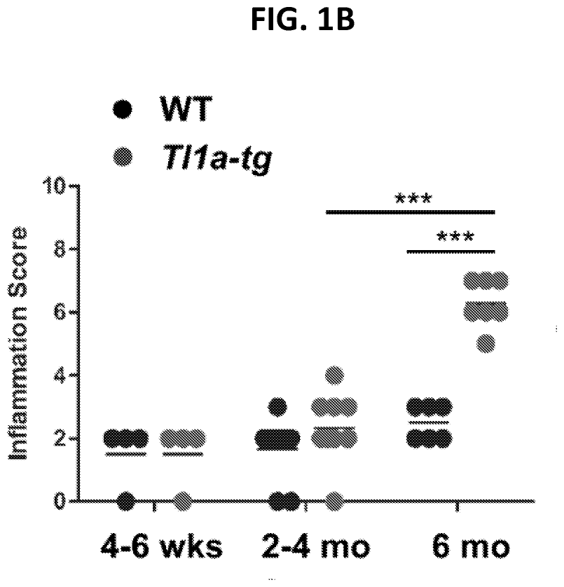

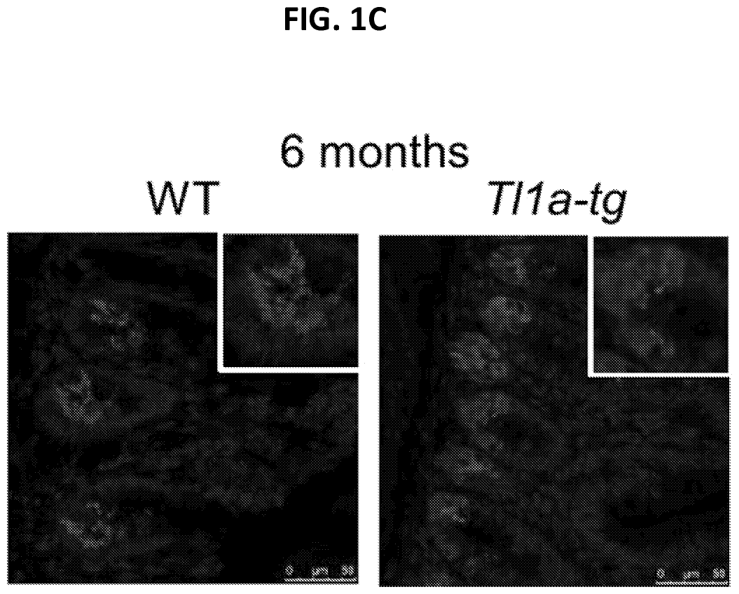

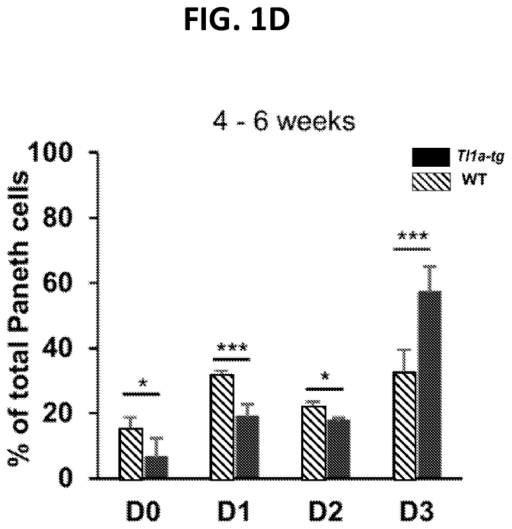

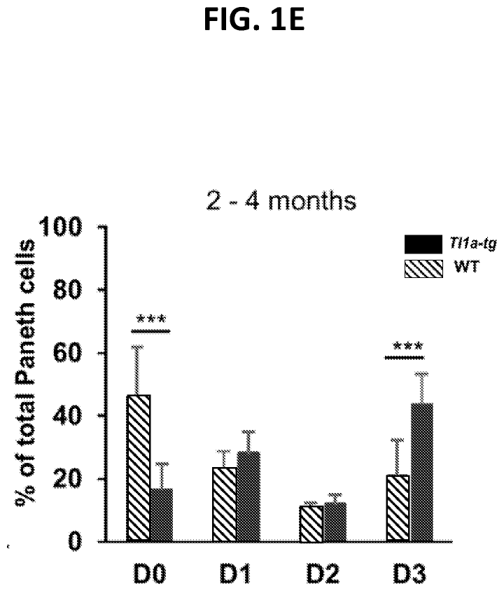

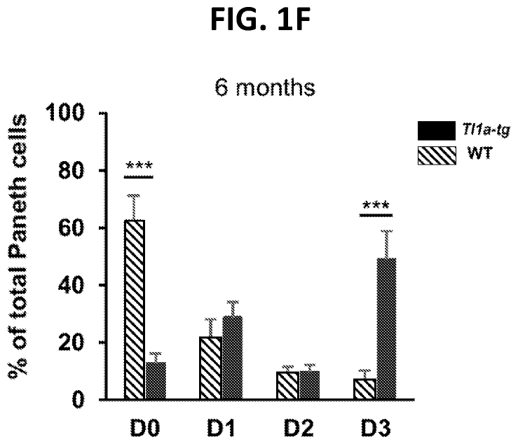

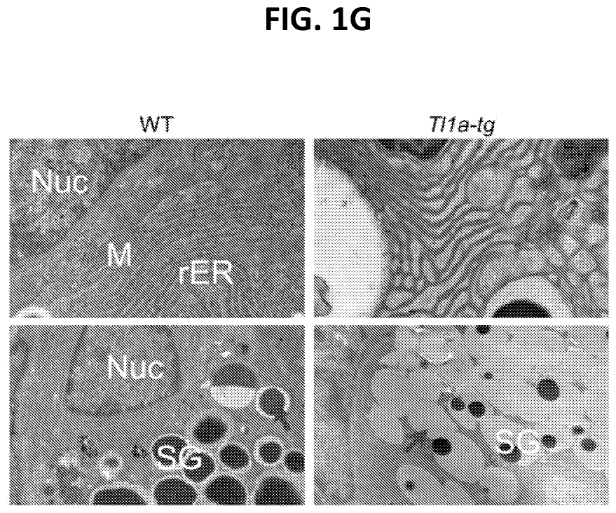

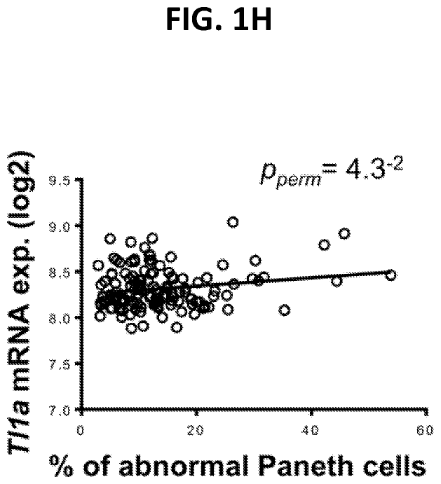

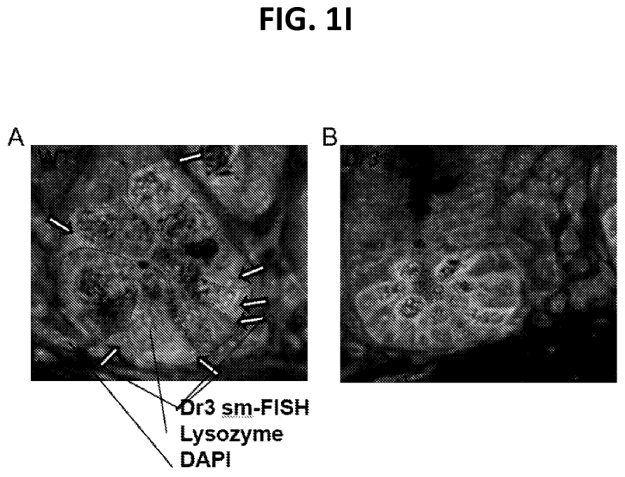

Paneth cell number, morphology, and degree of ileal inflammation in WT and Tl1a-tg mice were analyzed. The mice were analyzed at different ages (e.g., 4-6 weeks, 2-4 months, and 6 months) as shown in FIGS. 1A-1B. Paneth cell hyperplasia was observed in Tl1a-tg mice that were 4-6 weeks of age when there were no signs of ileitis (FIGS. 1A-1B). As the mice aged, the number of Paneth cells increased in both WT mice and Tl1a-tg mice; however, Tl1a-tg mice, when compared to WT mice, had significantly higher numbers of Paneth cells per crypt at any time-point. In mice older than 6 months of age, ileitis (inflammation) was observed in Tl1a-tg mice; however, ileitis was not observed in WT mice (FIG. 1B).

Paneth cell abnormalities in the intracellular distribution of granules containing antimicrobial proteins such as lysozymes have been implicated in a subset of Crohn's Disease (CD) patients and mice with genetic deletions in the autophagy and ER stress pathways. As shown in FIG. 1C, marked changes in Paneth cell morphology in 6-month-old Tl1a-tg mice were observed with a dominant diffuse lysozyme staining compared to normal granule packaging in WT mice.

Immunofluorescence staining of lysozyme: Lysozyme staining of ileal tissue sections was performed as described. In brief, formalin-fixed, paraffin-embedded tissue sections were deparaffinized, blocked with hydrogen peroxide, and boiled in citrate buffer (10 mM Sodium Citrate, 0.05% Tween-20; pH 6.0) for antigen-retrieval. Tissue sections were stained overnight at 4° C. with a goat polyclonal anti-Lysozyme antibody (Santa Cruz Biotechnology, CA; sc-27958) at a 1:150 dilution and followed by secondary antibody Alexa Fluor® 594-conjugated donkey anti-goat IgG (Molecular Probes, A-11058) for 2 hours at room temperature at a 1:500 dilution. Images were captured with a Leica TCS spectral microscope. Total numbers of Paneth cells and percentages of Paneth cells with DO-D3 features in ileum were quantitated by an investigator blinded to the mouse genotypes as described. For each mouse, Paneth cells were analyzed in well-oriented crypts from the ileum and the percentages of D0, D1, D2, D3 Paneth cells were determined as percentage of total numbers of Paneth cells. Paneth cells were counted to determine the average number of Paneth cells per crypt in each mouse.

A previously established scoring system was used to identify and quantify Paneth cells with abnormal lysozyme staining in the mice at different ages. FIGS. 1D-1F show the percentage of total Paneth cells with D0, D1, D2, and D3 features in WT and Tl1a-tg mice at indicated mice ages (e.g., 4-6 weeks, 2-4 months, and 6 months). In 4-week-old to 6-week-old WT mice, Paneth cells were observed with different degrees of granule abnormalities that shifted to a predominant DO phenotype characterized by normal granule size, number, and distribution upon aging of the mice. In contrast, 4-week-old to 6-week-old Tl1a-tg mice predominantly displayed Paneth cells with D3 abnormalities (diffuse lysozyme staining), which was also apparent in older Tl1a-tg mice. In comparison to WT mice, Tl1a-tg mice displayed a significantly higher degree of Paneth cell abnormalities at any age and these abnormalities preceded the development of ileitis. The degree of Paneth cell abnormalities in WT and Tl1a-tg mice was most pronounced in older mice (FIG. 1F). These results suggest that overexpression of TL1A drove changes in Paneth cell numbers and morphology preceding the development of ileitis.

Transmission electron microscopy was used to confirm Paneth cell abnormalities in Tl1a-tg mice.

Electron transmission microscopy: Ileal tissue fragments from age- and gender-matched WT and tl1a-tg mice were handled by standard methods to be fixed with 2.5% glutaraldehyde, 2.0% paraformaldehyde in 0.1 M sodium cacodylate buffer, and pH 7.4. After buffer washes, the samples were post-fixed with 1% OsO₄ in 0.1 M sodium cacodylate for 1 hour at room temperature, rinsed in dH₂O and incubated with 2% uranyl acetate at RT for 1 hour. After dehydration through a graded ethanol series, samples were infiltrated with Epon resin and embedded in freshly prepared Epon resin. After polymerization at 60° C. for 48 hours, samples were sectioned and placed on copper grids. 75-nm-thick sections were stained with uranyl acetate and lead citrate and examined with a JEOL 100CX transmission electron microscope at 60 k V operating voltage. Images were captured by an AMT digital camera (Advanced Microscopy Techniques Corporation, model XR611) with the assistance of the University of California Los Angeles Electron Microscopy Core Facility (UCLA Brain Research Institute).

As shown in FIG. 1G, WT mice displayed a well-organized ER structure and 7-month-old Tl1a-tg mice displayed disordered and distended ER and mitochondria, as well as increased vesiculation. FIG. 1G shows the representative transmission electron microscopy images of WT and Tl1a-tg Paneth cells where Nuc=nucleus; SG=secretory granules; M=mitochondria; rER=rough endoplasmic reticulum; and arrows indicate high-density secretory granules. Moreover, in Tl1a-tg mice, more Paneth cell granules were observed with less electron dense content surrounded by expanded electron-lucent halos formed by packaging of Muc2 mucin, potentially representing a degranulation or defect in the packaging of electron dense contents into granules. The data suggest that Paneth cells in Tl1a-tg mice had defective intracellular protein trafficking.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, ** p<0.005.

### Example 2: Association of Mucosal TL1A mRNA Expression in Uninflamed Ilea of CD Patients with Paneth Cell Abnormalities

A TNFSF15 risk haplotype associated with increased expression of TL1A in peripheral monocytes was previously identified. To determine the human relevance of the observed findings from murine TL1A over-expressing mice, the degree of Paneth cell abnormalities in post-surgical tissue sections of uninflamed ilea were analyzed in a 132 cohort of genotyped CD patients.

The association of TL1A mRNA expression with the percentage of Paneth cell abnormalities were analyzed in a dataset of 132 CD patients. Quantitative trait associations were performed between TNFSF15 gene expression and various Paneth cell phenotypes (e.g., D0, D1, D2, D3, D4, D1234 in %) using first two principal components in genotype data and sex as covariates. Associations with D1, D3 and D1234 (e.g., the abnormal Paneth cell phenotypes) were found to be significant, as shown in FIG. 1H and Table 1. The associations were confirmed by permutations and report a permuted p-value. Addition of ATG16L1 T300A (rs224 1880) as a covariate did not affect the TNFSF15 mRNA association with Paneth cell phenotypes. FIG. 1I shows DR3 expressed on murine ileal Paneth cells; where A) DR3 expression on ileal Paneth cells was detected by single-molecule fluorescent in situ hybridization (smFISH); Ileal tissue sections of WT small intestine were hybridized with smFISH probes against DR3 (red), stained with anti-lysozyme antibody (green), and counterstained with DAPI (blue); Expression of DR3 by Paneth cells is indicated by arrows; and B) Dr3-/-mice do not express DR3.

The data suggests that TL1A-driven Paneth cells abnormalities also occur in human CD and are an ATG16L1 T300A independent association.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, p<0.005.

TABLE 1
Association between Paneth cell phenotype
and mucosal TL1A expression

| | | PC | | | | |
|---|---|---|---|---|---|---|
| N | Gene | Phenotype | beta | p-value | N_perm | p_perm |
| 132 | TNFSF15 | D1 | 1.63 | 1.49E−03 | 10000 | 2.40E−03 |
| 132 | TNFSF15 | D3 | 4.63 | 7.14E−03 | 10000 | 9.60E−03 |
| 132 | TNFSF15 | D1234 | 6.89 | 4.35E−02 | 1000 | 4.30E−02 |

D0 = normal, D1, D3, = different abnormal Paneth cells; D1234 = all degrees of abnormal Paneth cell phenotypes.

### Example 3: Death Receptor 3 (DR3) Expression on Murine and Human Ileal Paneth Cells

The expression of the TL1A receptor, Death receptor 3 (DR3), on Paneth cells was investigated to determine if the effects of TL1A overexpression on Paneth cell hyperplasia and morphology is directly mediated. A single-molecule fluorescent in situ hybridization (smFISH) for DR3 was used.

smFISH: Tissues were prepared as described for immunofluorescence stainings. The DR3 transcripts were stained with RNAscope Multiplex Fluorescent Reagent Kit v2 (ACD, Newark, CA) according to manufacturer's instruction followed by immunofluorescence stainings for E-cadherin and lysozyme. The probe for murine Dr3 transcripts was custom designed to hybridize between exons 2 and 5 of the murine Dr 3 which is deleted in the process of the generation of Dr3^{-/-} mice. The probe for human Dr3 was purchased from Advanced Cell Diagnostics (Catalog number: 448571). Probe for PPIB (Cyclophilin B) and Hela cell pellets were used as a positive control in this assay and a probe for DapB was used as a negative control.

The smFISH for DR3 was used and co-stained with anti-lysozyme antibodies to identify Paneth cells within ileal crypts (FIG. 1I, at A). Expression of DR3 mRNA in lysozyme-positive Paneth cells in WT mice was observed (FIG. 1I, at A). Expression of DR3 in Dr3--mice demonstrating specificity of the DR3 probe was not observed (FIG. 1I, at B). Expression of DR3 in ileal tissue sections from CD patients and observed distinct expression on lysozyme-positive Paneth cells and enterocytes was confirmed.

### Example 4: Impact of the TL1A-DR3 Signaling Pathway on Paneth Cell Morphology

Based on the observation that DR3 is expressed on Paneth cells, it was hypothesized that TL1A may directly affect Paneth cell function.

Mice were generated with a specific deletion of DR3 in Paneth cells (Dr3ΔPC) by crossing floxed DR3 mice with the Defa6-Cre line. Dr3^{ΔPC} mice were then crossed to Tl1a-tg mice (Tl1a-tg Dr3APC mice) to assess direct and indirect effects of TL1A on Paneth cell morphology and function.

Mice: Mice containing a floxed Dr3 allele (Dr 3/1/f) were generated by GenOway. Defa6-Cre were provided by Richard Blumberg (Brigham and Women's Hospital, Boston, MA). Defa6-Cre were crossed to Dr3^{flox/flox} to generate Paneth-cell-specific deletion of DR3 (Dr3ΔPC). Tl1a-tg mice were crossed to Dr3^{ΔPC} to generate Tl1a-tg Dr3^{ΔPC}. All mice were maintained under specific-pathogen-free (SPF) conditions and handled according to the guidelines and approved protocols of the CSMC Animal Care and Use Committee.

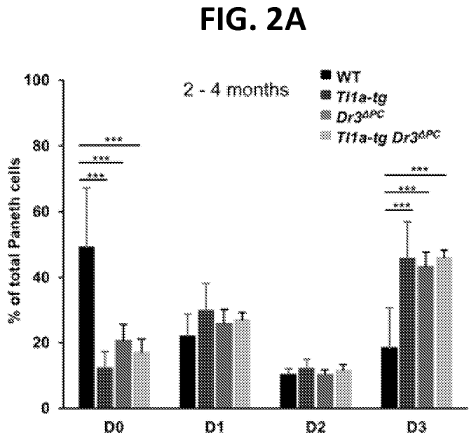

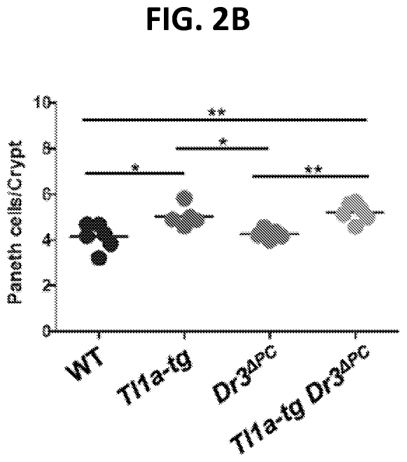

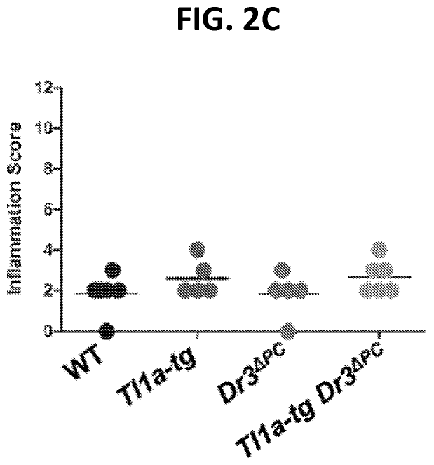

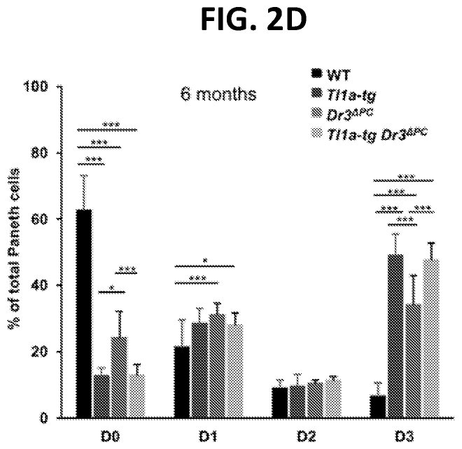

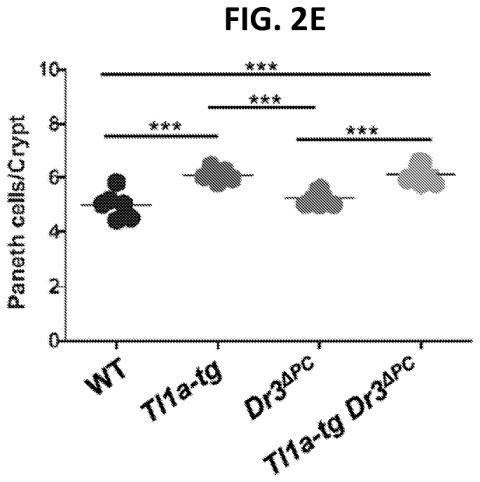

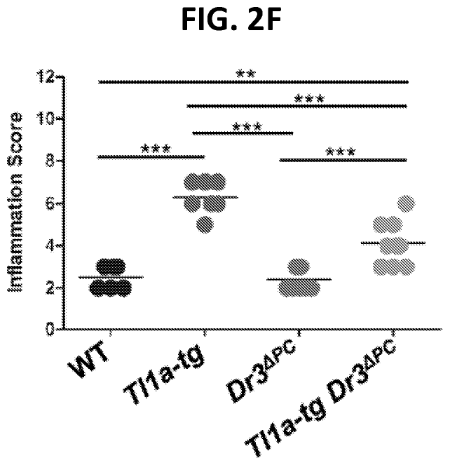

In comparison to WT mice, Dr3^{ΔPC} and Tl1a-tg Dr3APC mice displayed a significantly higher degree of Paneth cell abnormalities at any age and these abnormalities preceded the development of ileitis (FIG. 2A and FIG. 2D). FIGS. 2A-2C show Paneth cell phenotype in WT, Tl1a-tg, Dr3^{ΔPC}, and Tl1a-tg Dr3^{ΔPC} mice 2 to 4 months of age. 5 to 10 mice were used per group. FIGS. 2D-2F show Paneth cell phenotype in WT, Tl1a-tg, Dr3ΔPC, and Tl1a-tg Dr3ΔPC mice 6 months of age. 5 to 10 mice were used per group. The degree of Paneth cell abnormalities was most pronounced in older mice in both the 2-4-month-old mice and the 6-month-old mice (FIG. 2A and FIG. 2D). Compared to Tl1a-tg Dr3APC mice, Paneth cell abnormalities were less severe in Dr 3APC mice at 6 months of age, suggesting that direct and indirect effects of TL1A overexpression on Paneth cells contribute to the abnormal morphology. In addition, similar Paneth cell numbers were observed in Dr3^{ΔPC} mice when compared to WT mice in the 2-month-old to 4-month-old mice and 6-month-old mice (FIG. 2B and FIG. 2E). In contrast, Tl1a-tg Dr3^{ΔPC} mice had significantly increased Paneth cell numbers when compared to WT mice and Dr3^{ΔPC} mice, suggesting that Paneth cell hyperplasia is driven by TL1A overexpression in a manner independent of DR3 expression on Paneth cells. Spontaneous ileitis was not observed in Dr3^{ΔPC} mice but was observed in 6-month-old Tl1a-tg Dr3APC mice (FIG. 2C and FIG. 2F). Compared to Tl1a-tg mice, the ileitis observed in 6-month-old tl1a-tg Dr3^{ΔPC} mice was less severe, suggesting that the TL1A-DR3 pathway contributes to ileitis in a Paneth cell-intrinsic and a Paneth cell-extrinsic manner (FIG. 2F). DR3 signaling on Paneth cells contributes to the maintenance of normal granule packaging in Paneth cells.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, p<0.005.

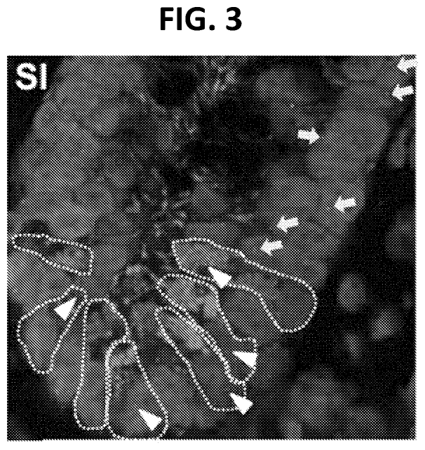

The expression of DR3 mRNA in non-inflamed small intestinal tissue sections was confirmed in a CD patient as shown in FIG. 3, where the DR3 mRNA probe is shown in green, the lysozyme is shown in pink, the E-cadherin is shown in red, the DAPI is shown in blue, the DR3 expression on Paneth cells are shown with arrowheads, and IECs are shown with arrows.

### Example 6: Global Gene Expression Analysis of Paneth Cells

The signaling pathways downstream of TL1A RNA-Sequencing analysis on Paneth cells from WT and Tl1a-tg mice were determined. Laser capture microscopy was used.

Laser Capture Microdissection for intestinal crypts, RNA-sequencing, and data analysis: The fixative for LCM is Methanol-Carnoy fixation containing 60% methanol, 30% chloroform and 10% acetic acid glacial. To compare the expression profile of Paneth cells between WT and tl1a-tg mice, 5 cells at the bottom of the well-oriented crypts were obtained through Leica LMD7000 Laser Microdissection Cell Capture System. RNA was extracted by Arcturus PicoPure® RNA Isolation Kit (ThermoFisher Scientific, Waltham, MA).

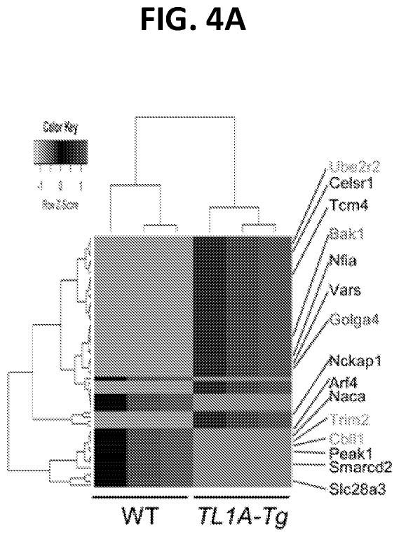

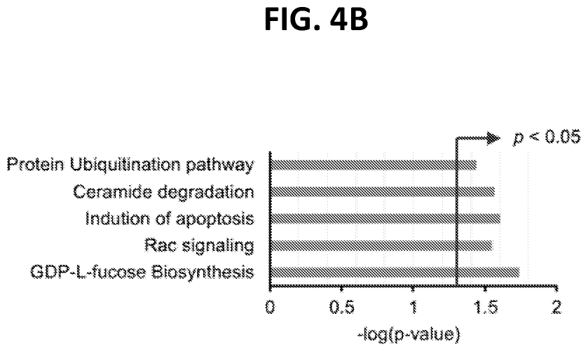

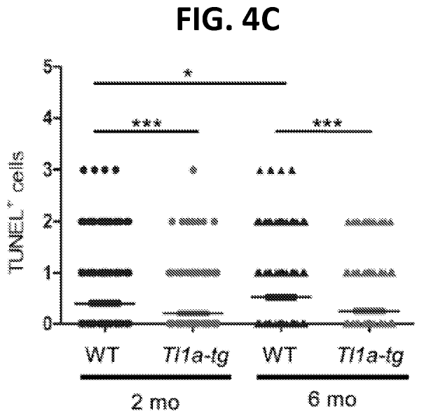

In brief, Paneth cell-containing intestinal crypts were isolated by laser capture microscopy on ileal sections from 8-week-old mice at a time point when no inflammation was observed. The 59 identified genes were differentially expressed in Tl1a-tg vs WT mice at adjusted p<0.01. FIG. 4A shows a heat map displaying the RNA sequencing data of the top 59 genes differentially expressed. The dendrograms shown to the left of the heat map and above the heat map represent hierarchical clustering of genes (shown as rows in FIG. 4A) and samples (shown as columns in FIG. 4A). The Golga4 gene was associated with cellular transports. The Ube2r2, Trim2, and Cbll1 genes were associated with protein ubiquitination. The Bak1 gene was associated with induction of apoptosis. FIGS. 4A-4C show transcriptional profiling of small intestinal crypts of untreated 2-month-old WT and Tl1a-tg mice. Three mice per group were used.

FIG. 4B shows the enrichment of pathways from genes differentially expressed in WT vs. tl1a-tg Paneth cells. Enrichment and ingenuity pathway analysis of differentially expressed transcripts revealed significant overrepresentation of biological processes of intracellular protein traffic, protein targeting, and localization (FIG. 4B). These results are consistent with Paneth cell granule packaging abnormalities in Tl1a-tg mice. Enrichment of transcripts encoding transporters and ligases, particularly those in protein ubiquitination pathways that are enriched in Atg16l1^{HM} mice were also observed. A significant up-regulation of apoptotic genes in Tl1a-tg mice, including the Bak1 gene, was also observed (FIGS. 4A-4B). The quantification of TUNEL⁺ cells in ileal crypts of 2-month-old and 6-month-old WT and Tl1a-tg mice is shown in FIG. 4C. At least 40 crypt-villus units were quantified per mouse. 5 to 6 mice per group were used. Data points represent individual crypts.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, ** p<0.005.

### Example 7: Association of Intestinal Microbiota, Paneth Cell Morphology, and Granule Maturation

The microbiome is relevant to inflammation in numerous diseases such as IBD. The effects of germ-free (GF) conditions on TL1A-mediated intestinal inflammation and Paneth cell abnormalities were evaluated.

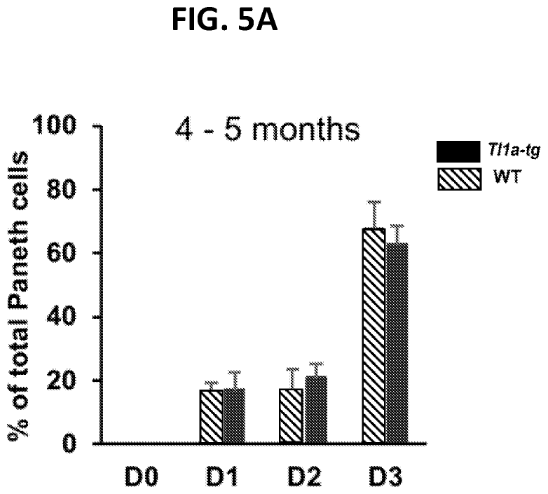

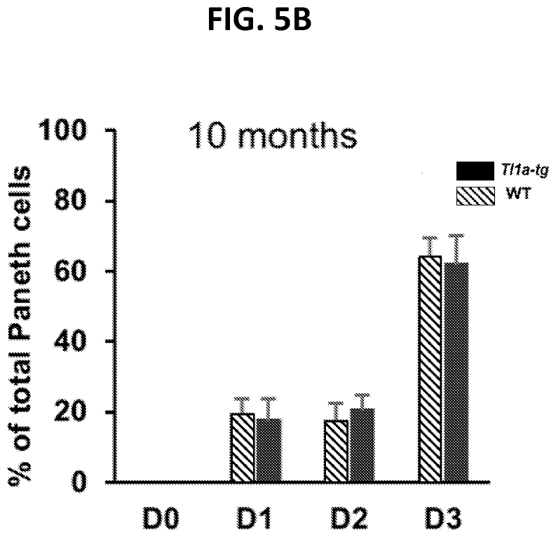

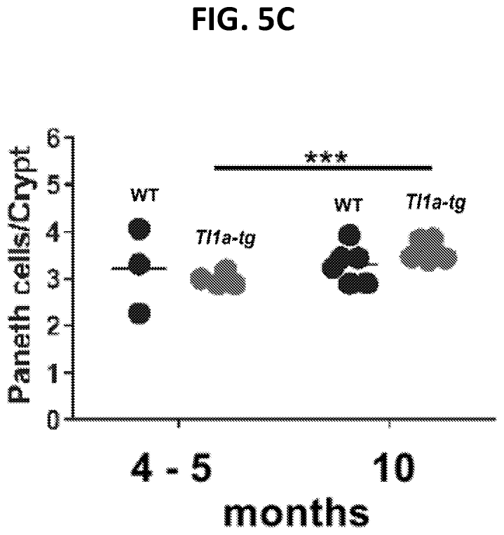

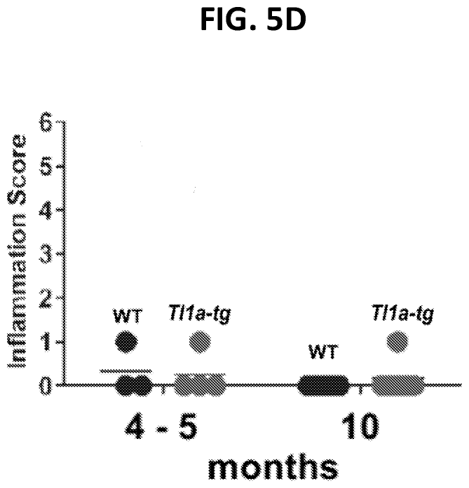

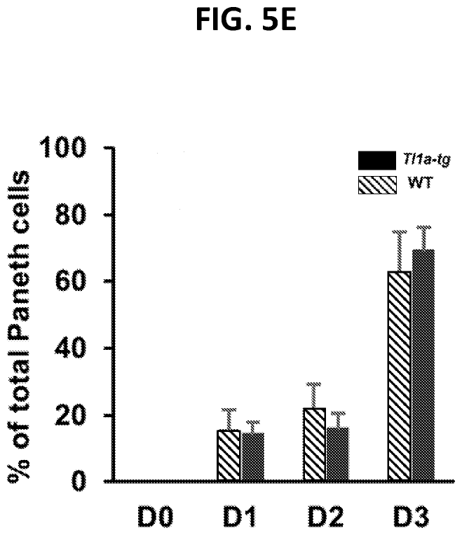

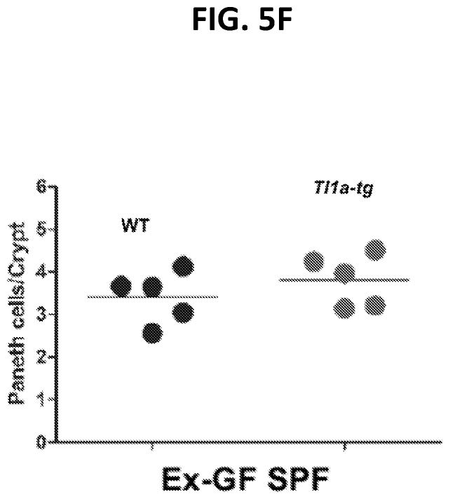

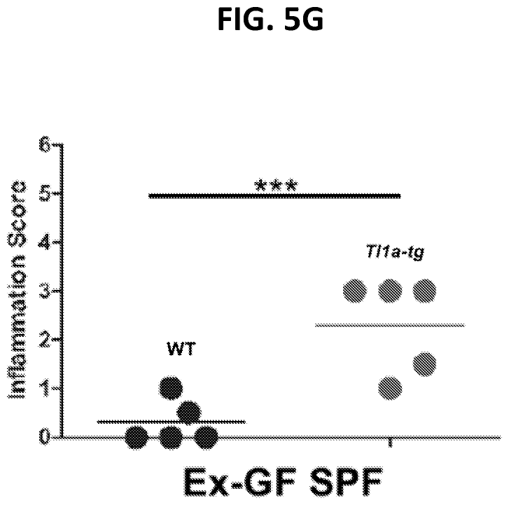

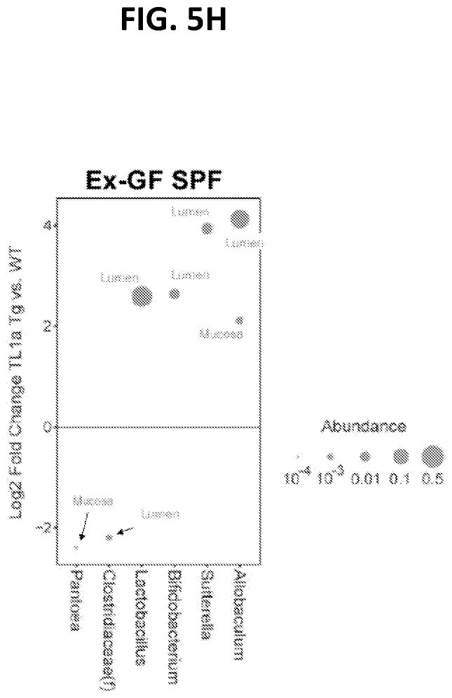

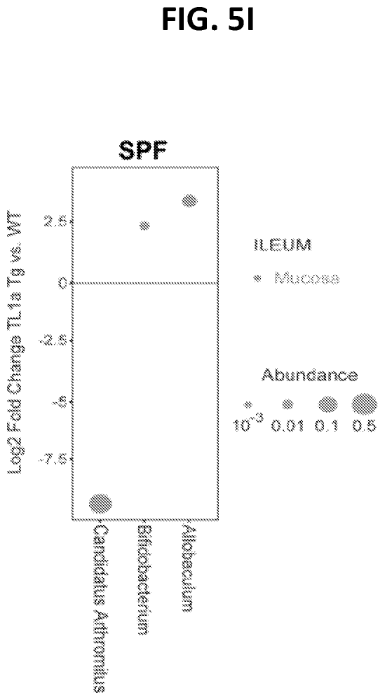

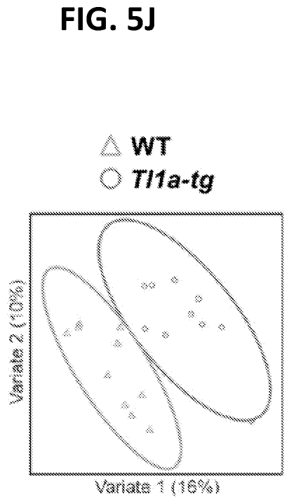

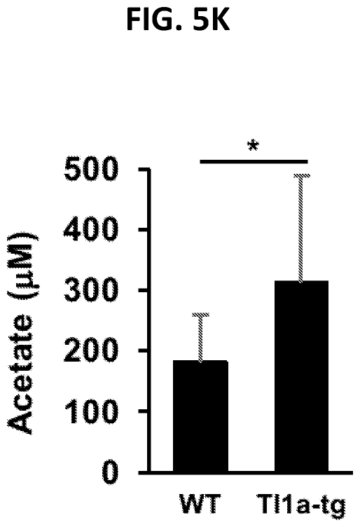

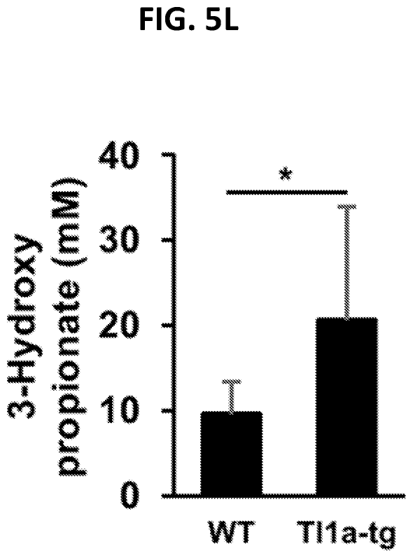

The absence of intestinal microbiota abrogated the spontaneous ileitis induced by TL1A overexpression, as there were no significant differences in ileal histopathology between GF Tl1a-tg mice and WT mice as shown in FIG. 5D. Around 5 to 10 mice were used per group. As shown in FIG. 5C, a significant increase in the number of Paneth cells in aged mice was observed in Tl1a-tg mice that was not observed in WT mice under GF conditions. However, a lack of Paneth cell granule maturation in WT mice and Tl1a-tg mice at 5 and 10 months of age with a D3-dominant Paneth cell phenotype in both genotypes was observed (FIGS. 5A-5B). The data suggests that commensal microbiota is required for the development of ileitis in Tl1a-tg mice, but dispensable for Paneth cell expansion. Furthermore, commensal microbiota is required for Paneth cell granule maturation in WT mice and Tl1a-tg mice.

### Example 8: Association of SPF Microbiota with Intestinal Inflammation, Paneth Cell Expansion, and Paneth Cell Granule Maturation

GF mice were reconstituted with murine SPF microbiota to determine if microbiota reconstitution can restore the development of ileitis and Paneth cell morphology. GF mice were reconstituted with SPF microbiota (Ex-GF SPF) at 2 months of age by oral gavage and inflammation. Paneth cell morphology was analyzed at 5 months of age.

Gnotobiotic and reconstituted mice: Tl1a-tg and WT mice were re-derived into germ-free (GF) status at the National Gnotobiotic Rodent Resource Center (University of North Carolina, Chapel Hill). 2-4 months old Tl1a-tg mice and WT littermates were orally gavaged with 200 μl of a 1:10 suspension of stool collected from Cedars-Sinai specific pathogen free (SPF) mice diluted in phosphate-buffered saline. GF mice were euthanized at 4-5 months or 10 months of age and reconstituted mice were euthanized at 5 months of age.

As shown in FIG. 5G, reconstitution with SPF microbiota restored the development of ileitis in Tl1a-tg mice. Around 3 to 6 mice were used per group. However, as shown in FIGS. 5E-5F, SPF reconstitution did not restore Paneth cell expansion or lead to Paneth cell granule maturation. FIG. 5E shows the percentage of total Paneth cells with D0, D1, D2, and D3 features in WT and Tl1a-tg mice at 10 months of age. FIG. 5F shows the number of Paneth cells per crypt in WT and Tl1a-tg mice with SPF microbiota (EX-GF SPF).

The observed data indicate that reconstitution of adult GF mice is sufficient to restore the development of ileitis in tl1a-tg mice, but insufficient for Paneth cell expansion and granule maturation.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, p<0.005.

### Example 9: Association of Abnormalities in Paneth Cell Morphology with Dysbiosis and Changes in Microbial Metabolites in Tl1a-Tg Mice

A 16S rRNA sequencing was performed.

16S rRNA sequencing and metabolomic analysis: Ileal luminal content was released by flushing with distilled deionized water then the mucosa-associated bacteria were released by DTT treatment according to published protocols. DNA extraction and sequencing of the 16S ribosomal RNA gene was performed for luminal and mucosal samples. In brief, bacterial DNA was extracted using the DNeasy PowerSoil kit (Qiagen) with bead beating. The V4 region of the 16S gene was amplified and barcoded using 515f/806r primers then 150×2 bp sequencing was performed on an Illumina HiSeq 2500. Raw data was processed in QIIME 1.9.1 and 97% operational taxonomic units (OTUs) were identified by closed reference OTU picking against the Greengenes database. The aqueous phase aliquots of homogenized luminal fecal samples in ddH₂O were sent to the West Coast Metabolomics Center, a NIH-supported core service located at UC Davis. These samples were analyzed using gas chromatography time of flight mass spectrometry and charged surface hybrid quadrupole time of flight mass spectrometry. In addition to these two untargeted pipelines, targeted gas chromatography quadrupole mass spectrometry analyses were performed to measure short chain fatty acids.

The 16SrRNA sequencing was performed to characterize the ileal microbiome of colonized ex-GF and SPF mice. The 16S rRNA sequencing was performed on luminal and mucosal ileal samples from littermate controls 2 months after reconstitution (where n=10/group). Differential microbial genera (or unclassified members of Clostridiaceae) were identified by negative binomial models. Log 2 fold change in abundance of various bacterial strains in Germ-free TL1a-tg and WT mice reconstituted with SPF microbiota is shown in FIG. 5H. The dot sizes represent microbial relative abundance. The sample types (e.g., lumen and mucosa) are also depicted and labeled in FIG. 5H. Reconstitution of adult GF mice with SPF flora was associated with higher abundance of luminal Lactobacillus, Bifidobacterium, Sutterella, and luminal and mucosa-associated Allobaculum in Tl1a-tg mice, suggesting that these bacterial strains might drive development of ileitis (FIG. 5H).

The abundance of microbial genera under SPF conditions in 2-month-old WT and Tl1a-tg mice was analyzed. A higher abundance of ileal, mucosal-associated Bifidobacterium, and Allobaculum in Tl1a-tg mice was observed and is shown in FIG. 5I. FIG. 5I also shows that the 16S rRNA sequencing was performed on luminal and mucosal ileal samples from 2-month-old SPF WT or tl1a-tg mice littermate controls (where n=10/group). Log2 fold change in abundance of various bacterial strains in SPF TL1a-tg and WT mice is shown in FIG. 5I.

Metabolic profiling of ileal mucosal washings of 2-month-old SPF WT and Tl1a-tg mice was performed. Differences in the metabolome profiles between WT and Tl1a-tg mice were observed with a significant increase in the concentration of the short-chain fatty acid acetate and the primary metabolite 3-hydroxy-propionate in Tl1a-tg mice (FIGS. 5J-5L). The observed increase in acetate is consistent with the increase of the abundance of the acetate producers Bifidobacterium and Allobaculum in Tl1a-tg mice. FIGS. 5J-5K show that the metabolites acetate and 3-hydroxy propionate were more abundant in the ileum of SPF Tl1a-tg mice than in WT mice. FIG. 5K shows the acetate and FIG. 5L shows the 3-hydroxy propionate.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, *** p<0.005.

### Example 10: Short-Chain Fatty Acid Acetate Drives Ileal Inflammation

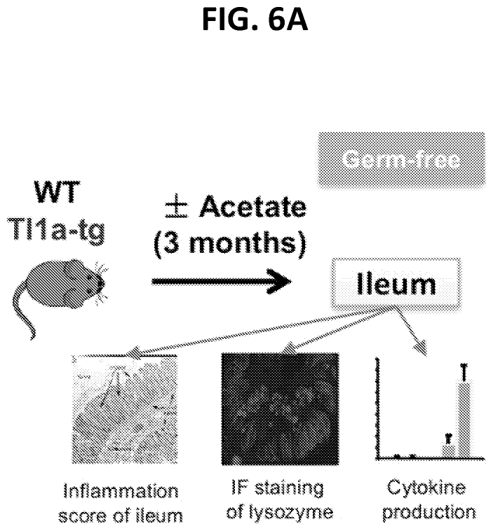

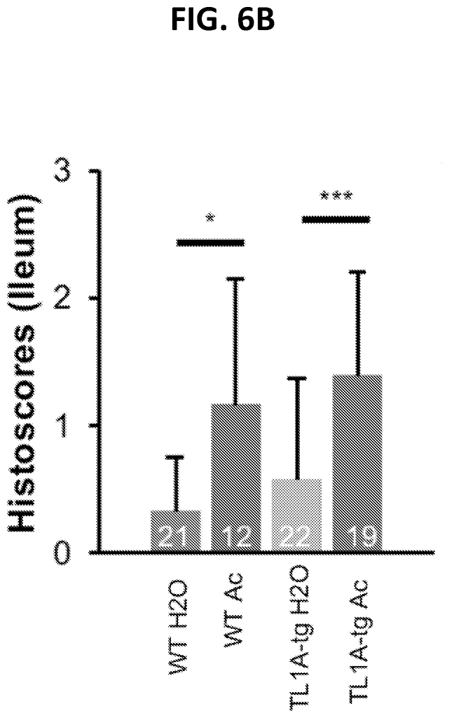

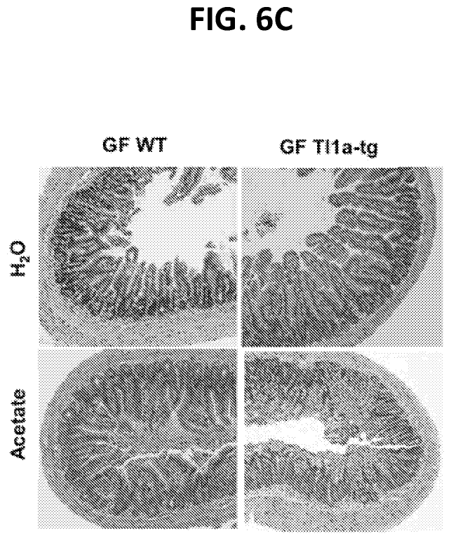

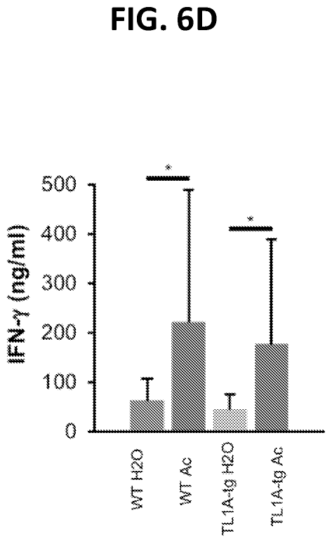

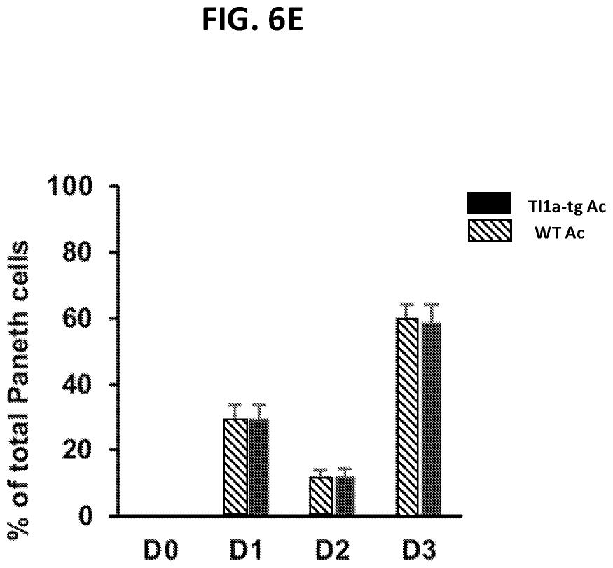

The impact of acetate on the development of ileitis was examined. FIG. 6A shows the experimental setup for acetate supplementation of germ-free mice.

Acetate supplementation: Sodium acetate (Sigma Aldrich) was added at 200 mM to drinking water. SPF mice were supplemented with acetate water for 2 or 4 months starting at 2 months of age. GF mice were supplemented with acetate water for 3 months starting at 2-3 months of age. Mice had ad libitum access to water and food during the entire treatment period.

12-week-old littermate GF WT mice or GF Tl1a-tg mice were treated with 200 mM of sodium acetate in drinking water (ad libitum) for 3 months. Both GF WT mice and GF Tl1a-tg mice that were supplemented with acetate developed ileal inflammation compared to mice receiving regular drinking water. FIG. 6B shows the histoscores of WT H2O mice, WT Ac (acetate) mice, Tl1a-tg H2O mice, and Tl1a-tg Ac mice. The number of mice used was 12 to 22 mice per group. FIG. 6C shows H&E (hematoxylin and eosin) images of the mice in the WT H2O, WT Ac, Tl1a-tg H2O, and Tl1a-tg Ac groups.

Isolation of mesenteric lymph nodes mononuclear cells, restimulation, and ELISA: Mesenteric lymph nodes (MLN) were removed from the peritoneal cavity and mesenteric fat was removed. Single cell suspension was prepared by crushing MLN between two glass slides and passing the cell suspension through a 40 μm cell strainer. Single-cell suspensions were restimulated with anti-CD3ε (0.5 μg/ml) and anti-CD28 (1 μg/ml) antibodies for 3 days. Cytokine levels in supernatants were measured by ELISA. Cytokine concentration in culture supernatants was assayed by ELISA for murine IFN-γ (eBioscience).

FIG. 6D shows the IFN-γ secretion of MLN of water or acetate treated GF WT or Tl1a-tg mice. The groups depicted include WT H2O, WT Ac (acetate), Tl1a-tg H2O, and Tl1a-tg Ac. Around 12-19 mice were used in each group. FIG. 6D shows significantly elevated IFN-γ secretion by MLN in acetate treated WT mice and acetate treated Tl1a-tg mice. However, as shown in FIG. 6E, acetate supplementation did not result in normal Paneth cell granule maturation, suggesting that acetate supplementation alone is not sufficient to drive normal Paneth cell granule development. FIG. 6E shows the percentage of Paneth cells with D0, D1, D2, and D3 features of water or acetate treated GF WT or Tl1a-tg mice. Around 6 mice were used per group. The observed data demonstrate that acetate is sufficient to drive ileitis in the absence of microbiota independently of the host genotype.

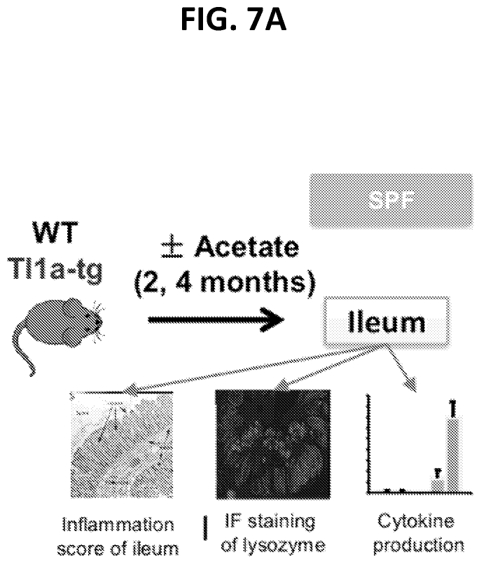

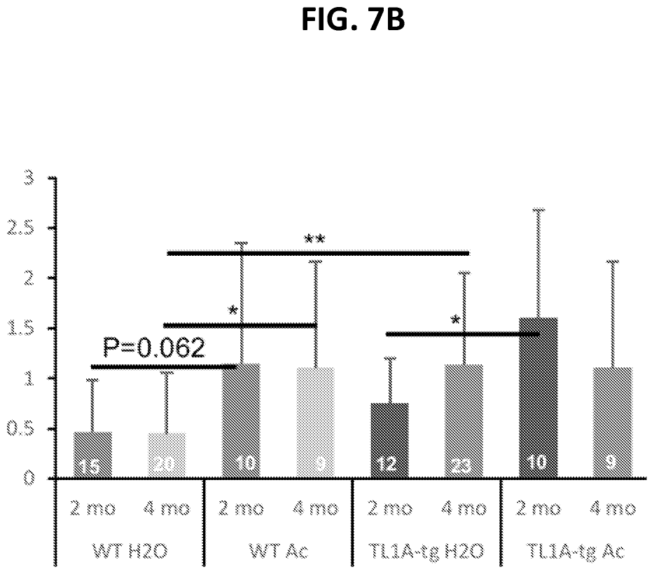

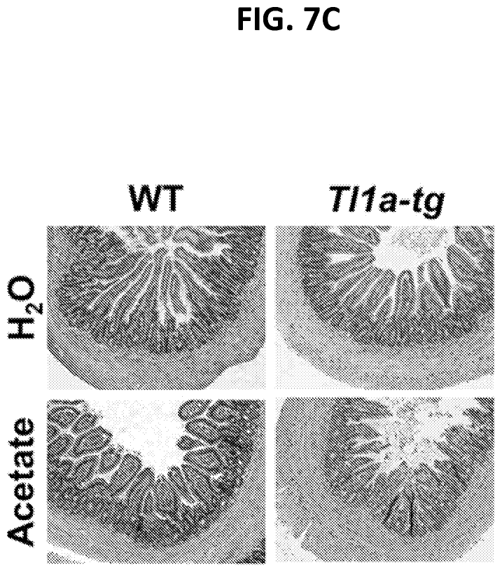

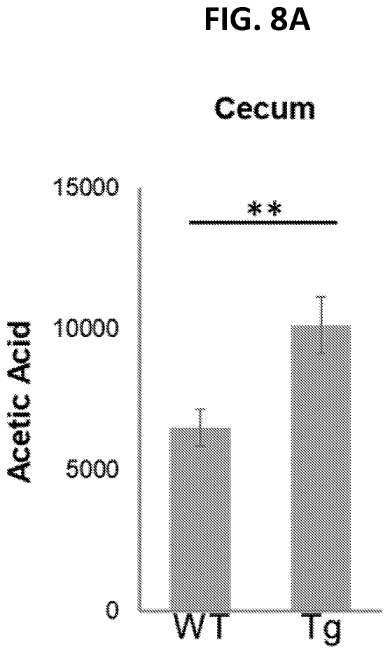

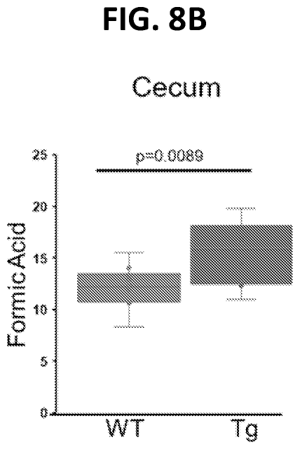

In a second set of experiments, SPF WT mice or tl1a-tg mice were treated with acetate in drinking water for 2 or 4 months starting at 2 months of age. The experimental setup is shown in FIG. 7A. WT mice treated with acetate for 4 months developed ileal inflammation compared to the water controls (FIG. 7B). Tl1a-tg mice that undergone acetate treatment for 2 months led to ileitis that was not observed in age-matched water control Tl1a-tg mice, suggesting an acceleration of the development of ileal inflammation in Tl1a-tg mice by acetate (FIG. 7B). 9 to 23 mice were used per group.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, p<0.005.

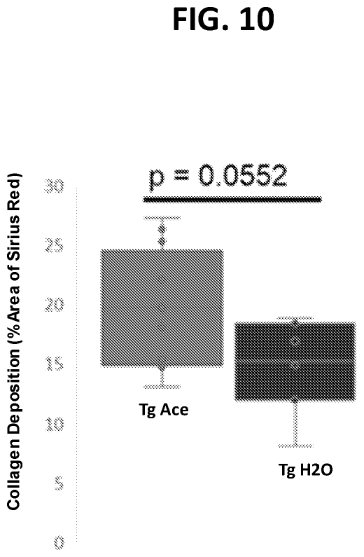

An increase in cecal collagen deposition was observed in Tl1a-Tg mice receiving acetate compared to mice receiving standard water (FIG. 10). The acetate induced cecal collagen deposition (fibrosis) in Germ-free IBD susceptible mice.

Data are presented as means±SD. Differences between groups were calculated using two-tailed Student t-test for two groups and by 1-way ANOVA with Tukey's HSD test for more than two groups. Differences were considered significant at p<0.05. * p<0.05, ** p<0.01, ** p<0.005.

1. A method of treating inflammatory bowel disease (IBD) in a subject, the method comprising: administering to the subject an active agent effective to reduce an amount of acetate or a metabolite thereof, or acetate producing bacteria in the subject, wherein the subject is predicted to develop ileitis, intestinal fibrosis, or colitis based, at least in part, on an increase in an amount of the acetate or the metabolite thereof, or an amount of the acetate producing bacteria, relative to a reference level in a reference subject that does not have the IBD detected in one or more samples obtained from the subject.

2. The method of claim 1, wherein the active agent comprises a small molecule drug, an antibody or antigen-binding fragment thereof, an inhibitor, an antibiotic, a probiotic, a prebiotic, an enzyme or catalytically active portion thereof, or any combination thereof.

3. (canceled)

4. (canceled)

5. (canceled)

6. (canceled)

7. (canceled)

8. (canceled)

9. (canceled)

10. The method of claim 1, wherein the active agent comprises a fecal microbiota transplant comprising a supplement derived from a stool sample from an individual that does not have the IBD to reduce an amount of the acetate producing bacteria in the subject.

11. (canceled)

12. (canceled)

13. (canceled)

14. (canceled)

15. The method of claim 1, wherein the active agent comprises an inhibitor, an antagonist of acetate receptor (GPR43), a chelating agent, or any combination thereof.

16. (canceled)

17. (canceled)

18. (canceled)

19. (canceled)

20. (canceled)

21. (canceled)

22. The method of claim 2, wherein the small molecule drug is therapeutically effective to disrupt the production of the acetate or the metabolite thereof in the subject by the acetate producing bacteria.

23. The method of claim 2, wherein the antibody or antigen binding fragment thereof is therapeutically effective to bind to the acetate or the metabolite thereof.

24. The method of claim 2, wherein the enzyme or the catalytically active portion thereof promotes the metabolism of acetate or the metabolite thereof in the subject.

25. The method of claim 24, wherein the enzyme comprises coenzyme A, acetyl-coenzyme A, formate acetyl transferase, or any combination thereof.

26. The method of claim 1, further comprising prescribing a change in a diet of the subject, wherein the change in the subject's diet comprises reducing the subject's intake of acid food, vinegar, acetic acid, carbohydrates, sugars, starches, probiotic-containing foods, kim chi, kombucha, or any combination thereof.

27. (canceled)

28. (canceled)

29. (canceled)

30. The method of claim 1, wherein the IBD is ulcerative colitis (UC), Crohn's disease (CD), or indeterminate colitis (IC).

31. (canceled)

32. (canceled)

33. The method of claim 1, further comprising administering to the subject a second agent, wherein the second agent comprises an inhibitor of Tumor necrosis factor (TNF)-like cytokine 1A (TL1A) activity or expression.

34. (canceled)

35. (canceled)

36. (canceled)

37. (canceled)

38. The method of claim 1, wherein the one or more samples comprises a stool sample, a blood sample, or a tissue biopsy, or any combination thereof.

39. The method of claim 1, wherein the subject is predicted to develop the ileitis, intestinal fibrosis, or colitis based, at least in part, on an increase in expression of a gene expression product of TNF superfamily member 15 (TNFSF15) in the one or more samples obtained from the subject relative to a reference expression level obtained from a reference subject without the IBD.

40. (canceled)

41. The method of claim 39, further comprising measuring the increase in expression of a gene expression product of TNFSF15 in the one or more samples obtained from the subject wherein the measuring comprises: (a) stimulating peripheral blood mononuclear cells (PBMCs) obtained from the one or more samples with immune complex under conditions sufficient for the PBMCs to express the gene expression product; (b) measuring the expression of the gene expression product; and (c) comparing the expression of the gene expression product with the reference expression level.

42. (canceled)

43. (canceled)

44. (canceled)

45. The method of claim 41, wherein the gene expression product of TNFSF15 is Tumor necrosis factor (TNF)-like cytokine 1A (TL1A), or messenger RNA (mRNA) encoding the Tumor necrosis factor (TNF)-like cytokine 1A (TL1A).

46. (canceled)

47. (canceled)

48. (canceled)

49. (canceled)

50. The method of claim 1, wherein the subject is predicted to develop the ileitis, intestinal fibrosis, or colitis based, at least in part, on detection of hyperplasia in a population of Paneth cells obtained from the one or more samples obtained from the subject, wherein the population of Paneth cells comprises a phenotype comprising D1, D3, D1234, or any combination thereof.

51. (canceled)

52. (canceled)

53. The method of claim 1, wherein the subject is predicted to develop the ileitis, intestinal fibrosis, or colitis based, at least in part, on an increase in a production or an amount of bile acids, interferon gamma (IFN-gamma), acetate or a metabolite thereof, acetate producing bacteria, 3-hydroxy-propionate, or any combination thereof, measured in the one or more samples obtained from the subject, relative to a reference level obtained from a reference subject without the IBD.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. The method of claim 1, wherein the subject is predicted to develop the ileitis, the intestinal fibrosis, or the colitis based, at least in part, on a presence of one or more genetic risk variants of tumor necrosis factor receptor superfamily member 15 (TNFRSF15) in the one or more samples obtained from the subject, wherein the one or more genetic risk variants is one or more single nucleotide polymorphisms (SNPs) comprising rs3810936, rs6478108, rs6478109, rs7848647, rs7869487, or any combination thereof.

69. (canceled)

70. (canceled)

71. (canceled)

72. (canceled)

73. (canceled)

74. (canceled)

75. (canceled)

76. (canceled)

77. The method of claim 1, wherein the subject is predicted to develop the ileitis, intestinal fibrosis, or colitis based, at least in part, on a reduction of protective short-chain fatty acids measured in the one or more samples relative to a reference level obtained from a reference subject without the IBD, wherein the protective short-chain fatty acids comprise butyrate, propionate, or a combination thereof.

78. (canceled)

79. (canceled)

80. A method of predicting a high likelihood that a subject with inflammatory bowel disease (IBD) will develop ileitis, fibrotic disease, colitis, or ulcerative colitis, the method comprising:
(a) measuring an increase in an amount of acetate or a metabolite thereof, or an amount of acetate producing bacteria in one or more samples obtained from the subject, wherein the increase in the amount of acetate or the metabolite thereof, or the acetate producing bacteria is relative to a reference level obtained from a reference subject without the IBD; and
(b) predicting the high likelihood that the subject with IBD will develop the ileitis, intestinal fibrosis, or colitis based, at least in part, on the increase in the amount of acetate or the metabolite thereof, or the acetate producing bacteria measured in (a).

81.-125. (canceled)
